(19) United States
(12) Patent Application Publication
(10) Pub. No.: US 2025/0268931 A1
(43) Pub. Date: Aug. 28, 2025

(54) TRANSCRIPTIONAL INHIBITOR FOR IMMUNE-RELATED GENE

(71) Applicant: The University of Tokyo, Tokyo (JP)

(72) Inventors: Ken ISHII, Tokyo (JP); Hideo NEGISHI, Tokyo (JP)

(73) Assignee: The University of Tokyo, Tokyo (JP)

(21) Appl. No.: 18/858,572

(22) PCT Filed: Apr. 21, 2023
(86) PCT No.: PCT/JP2023/015894

§ 371 Date: Oct. 21, 2024

(30) Foreign Application Priority Data
Apr. 22, 2022 (JP) 2022-070764

(51) Int. Cl. A61K 31/787; A61K 47/59

(52) U.S. Cl. CPC A61K 31/787; A61K 47/595

(57) ABSTRACT

It is an object of the present invention to provide a pyrrole imidazole polyamide (PIPA), which suppresses the expression of cytokines essential for activation of immune response, in particular, the expression of type I IFN, at a transcription stage. More specifically, the present invention relates to a PIPA that specifically binds to the binding region of an IRF (IFN regulatory factor) family transcription factor. Specifically, the present PIPA is a PIPA that binds to 5′-GAAAGTG-3 (SEQ ID No: 1) or a modified sequence thereof, 5′-GAAAG-3′ (SEQ ID No: 9) or a modified sequence thereof, 5′-GAAAA-3′ (SEQ ID No: 15) or a modified sequence thereof, or 5′-GAAAC-3′ (SEQ ID No: 21) or a modified sequence thereof.

### TECHNICAL FIELD

The present invention relates to a means for suppressing transcription of an immune-related gene. More specifically, the present invention relates to a pyrrole imidazole polyamide (PIPA), which specifically binds to the binding region of an IRF family transcription factor on genomic DNA and can function as a competitive pseudo-transcription factor.

### BACKGROUND ART

The immune system has the ability to distinguish between a self structure and a non-self structure at a molecular level. The immune system identifies a pathogen that invades a body as a non-self structure and activates an immune response, so that the immune system eliminates the pathogen through various mechanisms. In other words, the immune response that is specific to non-self structures and has an appropriate strength necessary for elimination is activated, and after elimination of the pathogens, the immune response is rapidly terminated, so that the homeostasis of a living body is maintained. However, it is known that the immune response does not always work beneficially for the living body, and that the failure in the control may lead to the onset or exacerbation of various diseases. First, activation of the immune response is accompanied by inflammation. Inflammation itself is an important response that causes accumulation and activation of immune cells, but excessive activation can destroy tissues and trigger various diseases called inflammatory diseases. In addition, the immune response is activated against non-self structures, but when this discrimination fails, the immune response is activated against self molecules, leading to autoimmune diseases. Moreover, when a pathogen that invades a cell, such as a virus, is eliminated, the virus and the cell are inevitably simultaneously eliminated. If such elimination response is prolonged without convergence, a critical situation is naturally caused for the living body. Furthermore, in situations other than diseases, in the case of mRNA vaccine, for example, if the mRNA vaccine excessively activates the antiviral response, the mRNA vaccine itself will be eliminated by the immune system, and thus, the vaccine cannot exhibit vaccine effects. As such, since the immune response has various disadvantageous aspects, it would be possible to treat or prevent various diseases and to enhance the effects of mRNA vaccine, etc., if the response can be controlled to an appropriate level.

Activation of the immune response is triggered by the direct encounter of immune cells with a pathogen, and further, various responses and cell species are continuously activated, so that cells of the innate immune system, which has low specificity but can be rapidly activated, first perform a defensive response, and then, a highly specific response is activated by cells of the adaptive immune system. Such activation of immune response and intercellular communication are mainly performed by liquid factors called cytokines, which play an important role in activation of the immune response, direction determination, and suppression of the immune response. That is to say, it has known that immune cells that encounter a pathogen produce various cytokines, which activate distant cells and also, depending on the type of cytokine, activate the response suitable for the pathogen. Thus, production of cytokines decreases when the pathogen is successfully eliminated, and the immune response is also terminated accordingly. On the other hand, if the pathogen is not eliminated completely, immune cells produce more cytokines, and the immune response is more strongly activated, resulting in a negative chain reaction, which leads to a cytokine storm in which a large amount of cytokines are released. As known from SARS-COV-2, cytokine storm causes lethal multi-organ failure due to excessive activation of the immune response (inflammatory response), leading to the death of the organism.

Various cytokines are involved in the control of immune response, and among others, type I IFN (interferon) is induced by recognition of pathogen components and viral infection, and plays an essential role in the activation of immune response (Non Patent Literature 1). Type I IFN consists of 13 subtypes including IFN-α and the like, in addition to IFN-β, IFN-ε, IFN-κ, and IFN-ω, all of which have an action to induce an antiviral state to cells and play an essential role in suppressing virus proliferation at a cellular level (Non Patent Literature 1 and Non Patent Literature 2). Furthermore, such type I IFN also has an action to activate various immune cells, and plays an important role in the body defense by activating the immune response as a whole through various functions such as promotion of the production of antibodies from B cells, induction differentiation into T cells, and maturation of dendritic cells (Non Patent Literature 3). On the other hand, as mentioned above, it is also known that type I IFN induces disadvantageous responses to the living body when the control thereof is disrupted, and thus, the type I IFN is an important cytokine that is deeply associated with autoimmune diseases and autoinflammatory diseases, such as rheumatoid arthritis, systemic lupus erythematosus and type I diabetes, deterioration of disease states during virus infection, cytokine storm, and inhibition of mRNA vaccine effects, and the like (Non Patent Literature 1, Non Patent Literature 4, and Non Patent Literature 9).

Production of cytokines such as type I IFN is triggered by the recognition of pathogen components by receptors called pattern recognition receptors (PRRs) in cells directly exposed to pathogens or pathogen components. When pathogen components are recognized by the pattern recognition receptors, intracellular signaling pathways are activated, ultimately causing activation of transcription factors. The activated transcription factors migrate to the nucleus and bind to the promoter region of a target gene, thereby releasing the region and promoting association of basic transcription factors, so that mRNA is produced from DNA serving as a template. The production of most cytokines is controlled at a transcriptional level, and after intracellular production of cytokine mRNA, it is translated into a protein and is further secreted out of the cell to exhibit its function. Cytokine transcription is usually controlled by multiple transcription factors, and the IFN regulatory factor (IRF) family transcription factors, NF-κB, and AP-1 are known to function as three major transcription factors downstream of the pattern recognition receptor (Non Patent Literature 1 and Non Patent Literature 5). All of these transcription factors are controlled by activation of signals downstream of the pattern recognition receptor, and are migrated to the nucleus after activation. It has been known that the dependence of a gene on each transcription factor is different depending on the gene type.

Among them, regarding IRF family transcription factors, 9 IRF transcription factors from IRF1 to IRF9 have been reported, and have been known as transcription factors that play an important role in, particularly, transcription of type I IFN (Non Patent Literature 5 and Non Patent Literature 6). In other words, it has been known that induction of type I IFN is extremely strongly dependent on IRF transcription factors, and it has been found that induction of type I IFN is significantly inhibited in cells or mice that delete IRF3 or IRF7, one of the IRF transcription factors. An IRF transcription factor-binding sequence (IRF-E) is present in all the promoter regions of the type I IFN gene, and when an IRF transcription factor specifically binds to this sequence, the expression of the type I IFN gene is activated (Non Patent Literature 7 and Non Patent Literature 8).

Further, recent reports have also revealed that the IRF transcription factor-binding sequence is present in the promoter regions of other cytokines such as IL-6 and TNF-α, and it has been elucidated that the IRF transcription factor is an important transcription factor for the control of not only type I IFNs but also various other cytokines (Non Patent Literature 5). It is considered that if such IRF transcription factors can be controlled, it will be possible to treat various diseases through the control of type I IFN response and immune response. However, it has been known that some IRF transcription factors among the 9 IRF transcription factors act in a complementary manner, and it has also been reported that the important IRF transcription factors are different in each cell and gene. Therefore, a method of specifically and effectively controlling the transcriptional activity of IRF transcription factors has not yet been developed, and this is one of the important issues to be solved in the field of immunology.

### CITATION LIST

### Non Patent Literature

Non Patent Literature 1: Fernandez-Ruiz and Niewold, J Invest Dermatol, doi: 10.1016/j.jid.2021.11.031 (2022).
Non Patent Literature 2: Gibbert et al., Br J Pharmacol 168, 1048-1058, doi: 10.1111/bph.12010 (2013).
Non Patent Literature 3: Ivashkiv et al., Nat Rev Immunol 14, 36-49, doi: 10.1038/nri3581 (2014).
Non Patent Literature 4: Jefferies, Front Immunol 10, 325, doi: 10.3389/fimmu.2019.00325 (2019).
Non Patent Literature 5: Negishi et al., Cold Spring Harb Perspect Biol. 10 (11): a028423. (2018).
Non Patent Literature 6: Tamura et al., Annu Rev Immunol. 26:535-84. (2008) Non Patent Literature 7: Braganca and Civas, Biochimie 80, 673-687, doi: 10.1016/s0300-9084 (99) 80021-2 (1998).
Non Patent Literature 8: Honda et al., Int Immunol 17, 1367-1378, doi: 10.1093/intimm/dxh318 (2005).
Non Patent Literature 9: Beuckelaer et al., Mol Ther. 24 (11): 2012-2020. (2016)

### SUMMARY OF INVENTION

### Technical Problem

In view of the above-described circumstances, it is an object of the present invention to provide a novel means for controlling transcription of an immune-related gene. More specifically, it is objects of the present invention: to provide a pyrrole imidazole polyamide (PIPA), which functions as a competitive pseudo-transcription factor specifically binding to the binding region of an IRF family transcription factor on genomic DNA, and suppresses transcription of the IRF family transcription factor; to provide a method for producing the PIPA; and to provide a cytokine transcription inhibitor comprising the PIPA.

### Solution to Problem

The present inventors have focused on the sequence of genomic DNA to which an IRF transcription factor binds, and have studied the use of PIPA as a means for suppressing the transcription of type I IFN. PIPA is a low-molecular-weight organic compound consisting of pyrrole and imidazole isolated from antibiotics, and is capable of binding to a specific genomic DNA sequence as a target. Furthermore, PIPA has the property of easily passing through the cell membrane. Therefore, if a PIPA that binds to an important sequence on a promoter with a certain binding force can be produced, it is possible to inhibit the binding of a transcription factor to the promoter region and to control the expression of a target protein at a transcription stage. Since the PIPA can be designed for any DNA sequence, it is theoretically possible to control the expression of all genes, and its introduction efficiency into cells is extremely high, compared with conventional gene regulation methods such as siRNA and antisense nucleic acids. However, identifying the sequence important for the binding of a transcription factor and designing a PIPA (e.g., adjusting the interval between pyrrole and imidazole) require a high level of specialized knowledge etc., and even if the binding sequence can be identified, it is not easy to design a PIPA with desired binding force and specificity. In fact, PIPAs that can effectively control activation of early immune responses, including type I IFN responses, have not yet been developed so far. The IRF family transcription factor is known to bind to specific sequence regions called IRF-E (IRF-responsive elements) and ISRE (IFN-stimulated response elements). Identification of these sequence regions is mainly based on the analysis results of artificial experimental systems at the cellular level. Therefore, among those sequences, it has not been narrowed down regarding which sequences are physiologically important and which sequences are appropriate as drug targets.

Thus, the present inventors have conducted intensive studies regarding the narrowing of the binding sequence of the IRF family transcription factor predicted from such sequence information and the designing of a PIPA that binds to the sequence with desired binding force and specificity. Specifically, the present inventors have narrowed down the sequences expected to be most suitable for the binding of the IRF family transcription factor to 4 sequences, have produced 4 PIPAs that each bind to these sequence, and have examined the influence of the PIPAs on IFN-transcription. As a result, it was confirmed that all of the PIPAs suppressed IFN-β transcription.

Grasping the aforementioned fact from another aspect, the PIPAs produced by the present inventors have a property by which the PIPAs binding to the sequence to which the IRF family transcription factors specifically bind, and it can be said that the PIPAs have a function as a “pseudo-transcription factor” that inhibits the transcription performed by the IRF family transcription factors via their binding sequences, and/or the activation thereof. Herein, in the present description, the term “pseudo-transcription factor” is broadly interpreted, and it is defined as a substance that has a property of binding to a sequence to which a specific transcription factor specifically binds, and inhibits the transcription performed by the transcription factors via their binding sequences, and/or the activation thereof.

The present inventors have elucidated for the first time that the PIPA according to the present embodiment, which is capable of functioning as a pseudo-transcription factor, can be used as a medicament for suppressing or inhibiting the transcription of a gene that is transcriptionally activated by an IRF family transcription factor and for suppressing excessive immune response, an adjuvant for mRNA vaccines, and the like, thereby completing the present invention.

Specifically, the present invention includes the following (1) to (38).
(1) A pyrrole imidazole polyamide (PIPA), which specifically binds to the binding region of an IRF (IFN regulatory factor) family transcription factor.
(2) The PIPA according to the above (1), which functions as a pseudo-transcription factor.
(3) The PIPA according to the above (1), which has a binding affinity of about 500 nM or less as a dissociation constant (Kd value) to the binding region.
(4) The PIPA according to the above (3), wherein the binding region is a region comprising 5′-GAAAGTG-3′ (SEQ ID No: 1) or a modified sequence thereof, and said modified sequence includes a sequence having a deletion or mutation of any one nucleotide in 5′-GAAAGTG-3′ (SEQ ID No: 1) or a sequence having an addition of one nucleotide to any site of 5′-GAAAGTG-3′ (SEQ ID No: 1).
(5) The PIPA according to the above (3), wherein the binding region is a region comprising 5′-GAAAG-3′ (SEQ ID No: 9) or a modified sequence thereof, and said modified sequence includes a sequence having a deletion or mutation of any one nucleotide in 5′-GAAAG-3′ (SEQ ID No: 9) or a sequence having an addition of one nucleotide to any site of 5′-GAAAG-3′ (SEQ ID No: 9).
(6) The PIPA according to the above (3), wherein the binding region is a region comprising 5′-GAAAA-3′ (SEQ ID No: 15) or a modified sequence thereof, and said modified sequence includes a sequence having a deletion or mutation of any one nucleotide in 5′-GAAAA-3′ (SEQ ID No: 15) or a sequence having an addition of one nucleotide to any site of 5′-GAAAA-3′ (SEQ ID No: 15).
(7) The PIPA according to the above (3), wherein the binding region is a region comprising 5′-GAAAC-3′ (SEQ ID No: 21) or a modified sequence thereof, and said modified sequence includes a sequence having a deletion or mutation of any one nucleotide in 5′-GAAAC-3′ (SEQ ID No: 21) or a sequence having an addition of one nucleotide to any site of 5′-GAAAC-3′ (SEQ ID No: 21).
(8) The PIPA according to any one of the above (4) to (7), which is represented by the following Formula (I), (II), (III) or (IV):

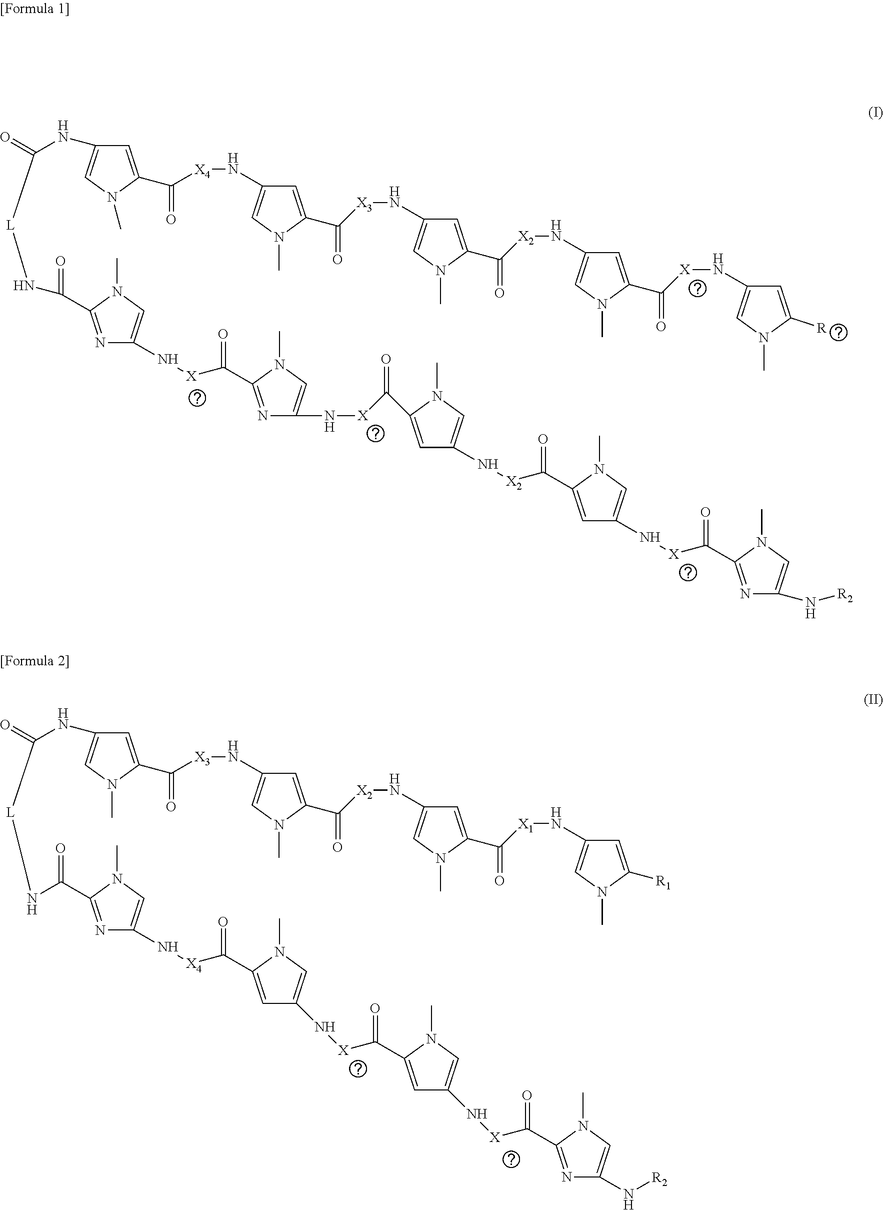

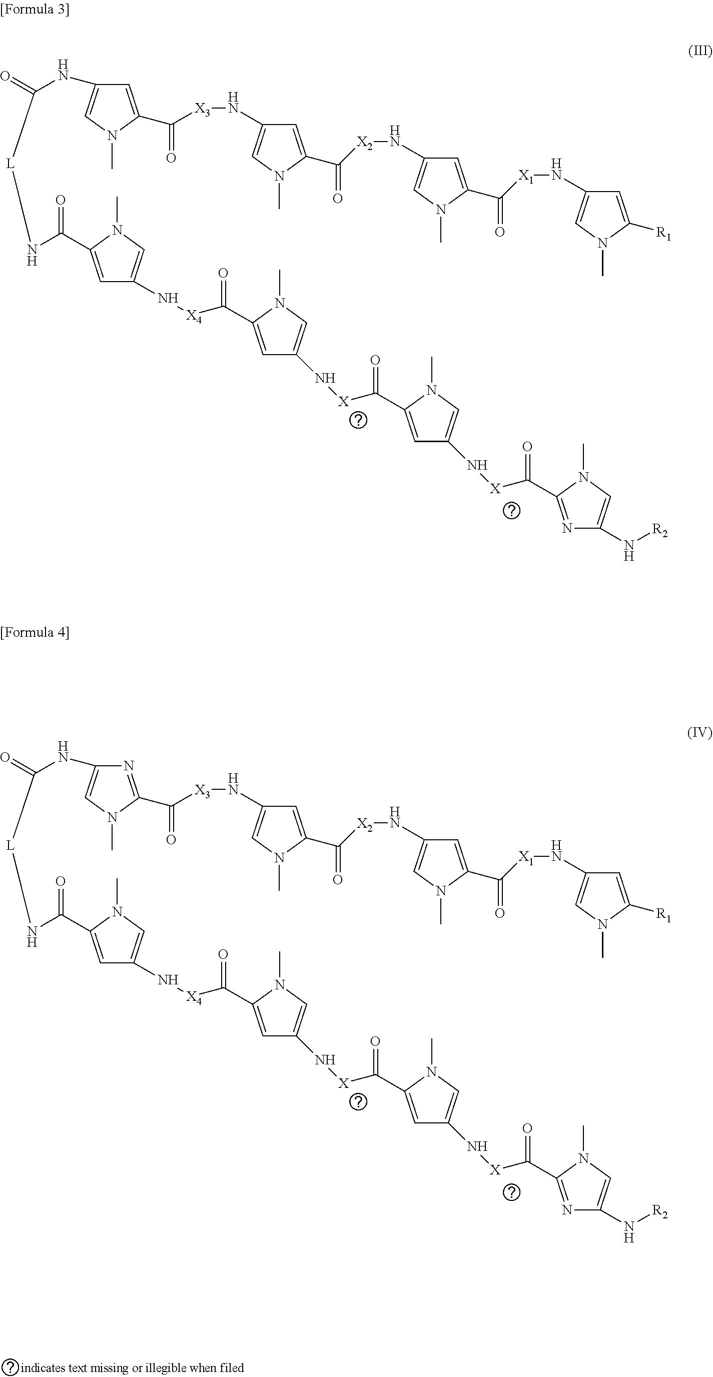

wherein in the Formulae (I), (II), (III) and (IV), L represents a C2 to 6 alkyl linker; R₁ and R₂ each represent a C1 to C10 alkyl group optionally having a substituent or a C1 to C10 alkyl group optionally containing an amide bond and/or an amino group, or R₁ and R₂ may bind to each other to form a C2 to 6 alkyl linker; and X₁, X₂, X₃, X₄, X₅, X₆, X₇ and X₈ identically or independently represent a bond or an aliphatic amino acid residue.
(9) The PIPA according to the above (8), which is represented by the following Formula (Ia), (IIa), (IIIa) or (IVa):

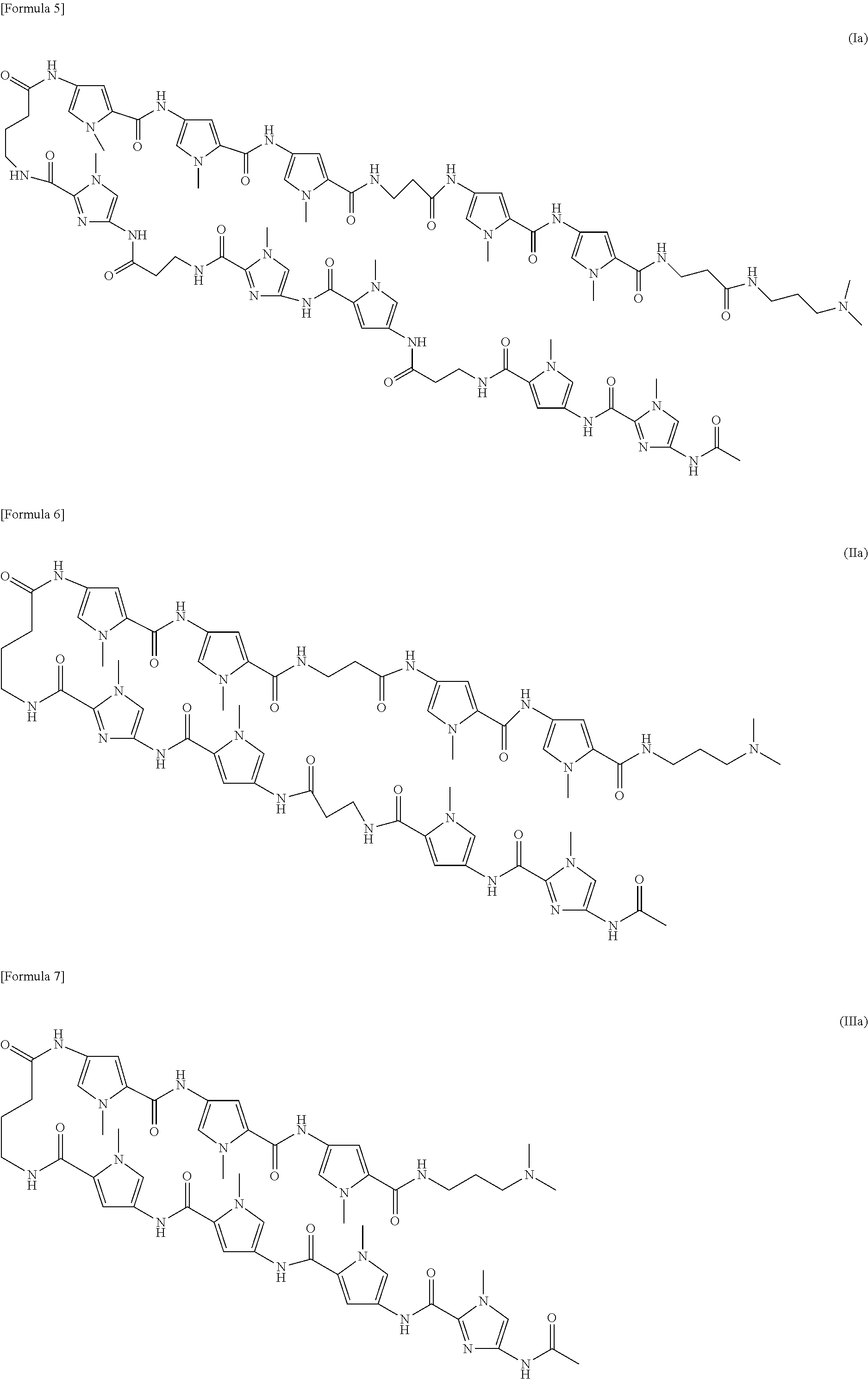

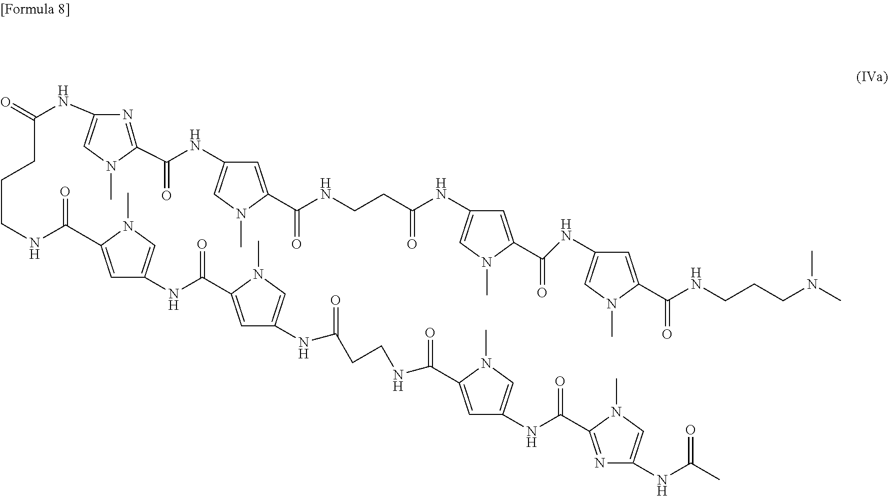 [SMILES: CC(=O)Nc1cn(C)c(C(=O)Nc2cc(C(=O)NCCC(=O)Nc3cc(C(=O)Nc4cc(C(=O)NCCCC(=O)Nc5cn(C)c(C(=O)Nc6cc(C(=O)NCCC(=O)Nc7cc(C(=O)Nc8cc(C(=O)NCCCN(C)C)n(C)c8)n(C)c7)n(C)c6)n5)n(C)c4)n(C)c3)n(C)c2)n1]

(10) A composition comprising a PIPA, for inhibiting the function of an IRF family transcription factor, wherein the PIPA specifically binds to the binding region of the IRF family transcription factor.
(11) The composition according to the above (10), wherein the PIPA is the PIPA according to any one of the above (2) to (7).
(12) The composition according to the above (10), wherein the PIPA is the PIPA according to the above (8).
(13) The composition according to the above (10), wherein the PIPA is the PIPA according to the above (9).
(14) A composition comprising a PIPA, for being used as a pseudo-transcription factor, wherein the PIPA specifically binds to the binding region of an IRF family transcription factor.
(15) An IRF family transcription factor inhibitor, comprising a PIPA that specifically binds to the binding region of the IRF family transcription factor.
(16) The IRF family transcription factor inhibitor according to the above (15), wherein the PIPA is the PIPA according to any one of the above (2) to (7).
(17) The IRF family transcription factor inhibitor according to the above (15), wherein the PIPA is the PIPA according to the above (8).
(18) The IRF family transcription factor inhibitor according to the above (15), wherein the PIPA is the PIPA according to the above (9).
(19) A medicament or a pharmaceutical composition for suppressing excessive immune response, wherein the medicament or the pharmaceutical composition comprises, as an active ingredient, a PIPA that specifically binds to the binding region of an IRF family transcription factor.
(20) The medicament or the pharmaceutical composition according to the above (19), wherein the PIPA is the PIPA according to any one of the above (2) to (7).
(21) The medicament or the pharmaceutical composition according to the above (19), wherein the PIPA is the PIPA according to the above (8).
(22) The medicament or the pharmaceutical composition according to the above (19), wherein the PIPA is the PIPA according to the above (9).
(23) An adjuvant, comprising a PIPA that specifically binds to the binding region of an IRF family transcription factor.
(24) The adjuvant according to the above (23), wherein the PIPA is the PIPA according to any one of the above (2) to (7).
(25) The adjuvant according to the above (23), wherein the PIPA is the PIPA according to the above (8).
(26) The adjuvant according to the above (23), wherein the PIPA is the PIPA according to the above (9).
(27) A method for producing an IRF family transcription factor inhibitor comprising a PIPA, wherein the method comprises a step of providing the binding region of an IRF family transcription factor, and a step of producing a PIPA specifically binding to the binding region.
(28) A conjugate, comprising a PIPA that specifically binds to the binding region of an IRF family transcription factor, and a PIPA auxiliary factor, wherein said PIPA auxiliary factor is a substance different from said PIPA, and said PIPA auxiliary factor is characterized in that it enhances the function of said PIPA, and/or it assists the action of the PIPA.
(29) The conjugate according to the above (28), wherein the PIPA and the PIPA auxiliary factor are linked to each other via a C1 to C6 linker.
(30) The conjugate according to the above (28), wherein the PIPA auxiliary factor is a substance that enhances the function of the PIPA, and is one or more substances selected from the group consisting of hydroxychloroquine, steroid, and FK506.
(31) The conjugate according to the above (28), wherein the PIPA auxiliary factor is a substance that assists the action of the PIPA, and is one or more substances selected from the group consisting of vitamin, cholesterol, and anisamide.
(32) The conjugate according to any one of the above (28) to (31), wherein the PIPA is the PIPA according to any one of the above (2) to (7).
(33) The conjugate according to any one of the above (28) to (31), wherein the PIPA is the PIPA according to the above (8).
(34) The conjugate according to any one of the above (28) to (31), wherein the PIPA is the PIPA according to the above (9).
(35) The conjugate according to any one of the above (28) to (31), which functions as a pseudo-transcription factor.
(36) The conjugate according to the above (32), which functions as a pseudo-transcription factor.
(37) The conjugate according to the above (33), which functions as a pseudo-transcription factor.
(38) The conjugate according to the above (34), which functions as a pseudo-transcription factor.

It is to be noted that the preposition “to” used in the present description indicates a numerical value range including the numerical values located left and right of the preposition.

### Advantageous Effects of Invention

According to the present invention, there is provided a PIPA, which specifically binds to the binding region of an IRF family transcription factor and suppresses the transcriptional activity thereof. The PIPA of the present invention exhibits an action to suppress the expression of various target genes including cytokines and chemokines whose transcription is activated by IRF family transcription factors, such as, for example, type I IFN, IL-6 and TNF-α. Therefore, a medicament and the like, comprising the PIPA of the present invention as an active ingredient, exhibit effects for prevention and treatment of excessive immune response caused by the target genes of the IRF family transcription factors, including these cytokines as typical examples, and diseases, etc., provoked by the excessive immune response.

### BRIEF DESCRIPTION OF DRAWINGS

FIG. 1 shows the results obtained by studying the influence of the PIPA according to the present embodiment on transcription of IFN-β. Raw264.7 cells, a macrophage cell line, were seeded on a 96-well plate at a cell density of 2×10⁴ cells/well, and were then culture for 8 hours for adhesion. Thereafter, IRF-PIPA was added to the cultured cells to final concentrations of 1, 3, or 10 μM. After completion of the culture for 24 hours, LPS was added to the cultured cells to a final concentration of 20 ng/mL, and the obtained mixture was then cultured for 16 hours, followed by the recovery of the supernatant. The supernatant was 2-fold diluted with 1% BSA-PBS, and the IFN-β concentration was then measured by ELISA.

FIG. 2 shows the results obtained by studying the influence of IRF-PIPA 3 on transcription of IFN-β, IL-6 and TNF-α (1). Peritoneal macrophages were harvested from wild-type mice, and were then seeded on a 96-well plate at a cell density of 2.5×10⁵ cells/well, followed by culturing for 2 hours. Thereafter, the cultured cells were washed twice with PBS. Subsequently, IRF-PIPA 3 was suspended in 10% FBS-RPMI or D-MEM to a final concentration of 3 or 10 UM, and was then added to the wells. After completion of the culture for 24 hours, Poly(I:C) was added to the cultured cells to a final concentration of 1 μg/mL, and the obtained mixture was then cultured for 16 hours. The supernatant was recovered, and the concentrations of IFN-β, IL-6, and TNF-α were then measured by ELISA.

FIG. 3 shows the results obtained by studying the influence of IRF-PIPA 3 on transcription of IFN-β, IL-6 and TNF-α (2). Using PBS, peritoneal macrophages were harvested from mice, and were then seeded on a 96-well plate at a cell density of 2.5×10⁵ cells/well, followed by culturing for 2 hours. Thereafter, the cultured cells were washed twice with PBS. IRF-PIPA 3 was suspended in a 10% FBS-RPMI or MEM medium to a final concentration of 6 μM, and the obtained suspension was then added to the cells. After completion of the culture for 24 hours, Poly(I:C) was added to the cultured cells to a final concentration of 1 μg/mL. The obtained mixture was cultured for 2, 4, or 6 hours, and the cells were recovered. Using TRIsure, the recovered cells were suspended at 100 L/well and was then recovered. Thereafter, the relative expression levels of Ifnb1, Il6, and Tnf genes were measured by qRT-PCR.

FIG. 4 shows the results obtained by studying the cytotoxic activity of IRF-PIPA 3. In order to verify the cytotoxic activity of IRF-PIPA 3, dsDNA concentration (left view in the figure) and LDH concentration (right view in the figure) in the culture supernatant were measured. The same experiment as that in FIG. 2 was carried out, and the culture supernatant was recovered. A lysate obtained by mixing 2.5×10⁵ peritoneal macrophages with 90 μL of 10% FBS-RPMI or D-MEM medium and 10 μL of Lysis Buffer, and then disrupting the cells, was used as a positive control. FIG. 5 shows the results obtained by studying the specificity and affinity of the binding of IRF-PIPA 3 to a target sequence. Using Octet K2, the binding of each concentration of IRF-PIPA 3 shown in the figure and a target DNA sequence was analyzed. After mixing a PIPA solution with target DNA, the binding of the two substances was measured for 60 seconds. Thereafter, the DNA sequence was transferred into a solution that did not contain PIPA, and the dissociation of the two substances was measured for further 60 seconds. From changes in the binding and dissociation at each concentration over time, a Kd value was calculated as a dissociation constant indicating a binding force.

FIG. 6 shows the results obtained by studying the influence of IRF-PIPA 3 on transcription of IFN-β, IL-6 and TNF-α in vivo. IRF-PIPA 3 was administered into the abdominal cavity of wild-type mice in an amount of 400 μg/mouse, and 3 hours after the administration, 250 μg/mouse Poly(I:C) was also administered into the abdominal cavity. Two hours after the administration of Poly(I:C), peritoneal macrophages were recovered, and the relative expression levels of Ifnb1, Il6, and Tnf genes were then analyzed by qRT-PCR.

FIG. 7 shows the results obtained by studying the influence of IRF-PIPA 3 on the expression of individual proteins IFN-β, IL-6 and TNF-α in vivo. IRF-PIPA 3 was administered into the abdominal cavity of wild-type mice in an amount of 400 μg/mouse, and 24 hours after the administration, 250 μg/mouse Poly(I:C) was also administered into the abdominal cavity. Two hours after the administration of Poly(I:C), blood was collected, and individual protein concentrations of IFN-β, IL-6 and TNF-α in the plasma were then measured by an ELISA method.

FIG. 8 shows the results obtained by studying the influence of IRF-PIPA 3 on activation of IFN-β transcription by an mRNA vaccine (an mRNA vaccine against SARS-COV-2). Peritoneal macrophages were harvested from wild-type mice, and were then seeded on a 96-well plate at a cell density of 2.5×10⁵ cells/well, followed by culturing for 2 hours. Thereafter, the cultured cells were washed twice with PBS. Subsequently, IRF-PIPA 3 was suspended in 10% FBS-RPMI or D-MEM to a final concentration of 3 or 10 UM, and was then added to the wells. After completion of the culture for 24 hours, an mRNA vaccine was added to the cultured cells to a final concentration of 1 μg/mL, and the obtained mixture was then cultured for 20 hours. Thereafter, RNA was prepared from the cells, and the relative expression level (left) of Ifnb1 gene was measured by qRT-PCR. Otherwise, after the recovery of a culture supernatant, the concentration (right) of IFN-β was measured by ELISA.

FIG. 9 shows the results obtained by studying the influence of IRF-PIPA 3 on induction of the expression of individual proteins IFN-β, IL-6 and TNF-α by an mRNA vaccine (mRNA encoding ovalbumin). Peritoneal macrophages were harvested from wild-type mice, and were then seeded on a 96-well plate at a cell density of 2.5×10⁵ cells/well, followed by culturing for 2 hours. Thereafter, the cultured cells were washed twice with PBS. Subsequently, IRF-PIPA 3 was suspended in 10% FBS-RPMI or D-MEM to a final concentration of 6 μM, and was then added to the wells. After completion of the culture for 16 hours, an mRNA vaccine was added to the cultured cells to a final concentration of 0.5 μg/mL, and the obtained mixture was then cultured for 20 to 24 hours. Thereafter, the culture supernatant was recovered, and each protein concentration of IFN-β, IL-6 or TNF-α was then measured by ELISA.

FIG. 10 shows results demonstrating that suppression of the expression of a vaccine antigen by double-stranded RNA can be improved by IRF-PIPA. IRF-PIPA 3 was administered at 400 μg/mouse into the muscle (i.m.) or abdominal cavity (i.p.) of wild-type mice, and 24 hours after the administration, an mRNA vaccine against SARS-CoV-2, manufactured by Moderna, was administered at 1 μg/mouse into the muscle of the mice. At the same time, 250 μg/mouse Poly(I:C) was administered into the abdominal cavity or muscle of the mice. One day after the administration, blood was collected from the mice, and the concentration of RBD in the plasma was then measured by an ELISA method.

### DESCRIPTION OF EMBODIMENTS

Hereafter, the embodiments for carrying out the present invention will be described. It is to be noted that when “the present embodiment” is mentioned, it refers to all embodiments described in the present description, unless otherwise indicated.

A first embodiment relates to a pyrrole imidazole polyamide (PIPA), which specifically binds to the binding region of an IRF (IFN regulatory factor) family transcription factor (hereinafter also referred to as “the PIPA according to the present embodiment”).

The “PIPA” of the present embodiment is a low-molecular-weight organic compound mainly comprising pyrrole (Py) and imidazole (Im) isolated from antibiotics, and is capable of binding to double-stranded DNA in a sequence-specific manner. PIPA penetrates into the minor groove of double-stranded DNA and reversibly binds with each base of the DNA via hydrogen bonds. Py has an action to bind to A, T, and C residues, whereas Im has an action to bind to G residues. By appropriately setting the sequences of Py and Im, PIPA can be designed to bind to any given DNA sequence. PIPA generally adopts a hairpin loop structure (see the specific structures of PIPAs shown below), such that Py/Im pairs can target C-G base pairs and Py/Py pairs can target A-T or T-A base pairs, in the minor group of double-stranded DNA. However, even if it is a pair of two independent PIPAs that are not connected with each other by a hairpin loop, it may adopt a ring structure. Thus, depending on purpose, an appropriate structure can be designed.

The PIPA of the present embodiment is a polyamide in which pyrrole and imidazole are mainly linked to each other by an amide (carboxamide) bond. In order to adjust the length or the degree of curvature, this amide chain may appropriately comprise aliphatic amino acid residues, for example, glycine, β-alanine, γ-aminobutyric acid, R2, 4-diaminobutyric acid and 5-aminovaleric acid, etc. Herein, γ-aminobutyric acid can be used to maintain the hairpin loop structure, and β-alanine can be used to optimize the hydrogen bonding between the adjacent amino acid portion and the nucleotide portion of the DNA chain.

The binding region of an IRF family transcription factor, to which the PIPA according to the present embodiment binds, comprises Sequence 1 to Sequence 4 or their modified sequences, as shown below.

### Sequence 1 and a Modified Sequence Thereof

A region comprising 5′-GAAAGTG-3′ (SEQ ID No: 1) or a modified sequence of the sequence shown in SEQ ID No. 1, wherein the modified sequence is a sequence having a deletion or mutation of any one nucleotide in 5′-GAAAGTG-3′ (SEQ ID No: 1), or a sequence having an addition of one nucleotide to any site of 5′-GAAAGTG-3′ (SEQ ID No: 1).

Examples of the above-described modified sequence may include 5′-NAAAGTG-3′ (SEQ ID No: 2), 5′-GNAAGTG-3′ (SEQ ID No: 3), 5′-GANAGTG-3′ (SEQ ID No: 4), 5′-GAANGTG-3′ (SEQ ID No: 5), 5′-GAAANTG-3′ (SEQ ID No: 6), 5′-GAAAGNG-3′ (SEQ ID No: 7), and 5′-GAAAGTN-3′ (SEQ ID No: 8) (provided that N is A, T, G or C).

### Sequence 2 and a Modified Sequence Thereof

A region comprising 5′-GAAAG-3′ (SEQ ID No: 9) or a modified sequence of the sequence shown in SEQ ID No. 9, wherein the modified sequence is a sequence having a deletion or mutation of any one nucleotide in 5′-GAAAG-3′ (SEQ ID No: 9), or a sequence having an addition of one nucleotide to any site of 5′-GAAAG-3′ (SEQ ID No: 9).

Examples of the above-described modified sequence may include 5′-NAAAG-3′ (SEQ ID No: 10), 5′-GNAAG-3′ (SEQ ID No: 11), 5′-GANAG-3′ (SEQ ID No: 12), 5′-GAANG-3′ (SEQ ID No: 13) and 5′-GAAAN-3′ (SEQ ID No: 14) (provided that N is A, T, G or C).

### Sequence 3 and a Modified Sequence Thereof

A region comprising 5′-GAAAA-3′ (SEQ ID No: 15) or a modified sequence of the sequence shown in SEQ ID No. 15, wherein the modified sequence is a sequence having a deletion or mutation of any one nucleotide in 5′-GAAAA-3′ (SEQ ID No: 15), or a sequence having an addition of one nucleotide to any site of 5′-GAAAA-3′ (SEQ ID No: 15).

Examples of the above-described modified sequence may include 5′-NAAAA-3′ (SEQ ID No: 16), 5′-GNAAA-3′ (SEQ ID No: 17), 5′-GANAA-3′ (SEQ ID No: 18), 5′-GAANA-3′ (SEQ ID No: 19), and 5′-GAAAN-3′ (SEQ ID No: 20) (provided that N is A, T, G or C).

### Sequence 4 and a Modified Sequence Thereof

A region comprising 5′-GAAAC-3′ (SEQ ID No: 21) or a modified sequence of the sequence shown in SEQ ID No. 21, wherein the modified sequence is a sequence having a deletion or mutation of any one nucleotide in 5′-GAAAC-3′ (SEQ ID No: 21), or a sequence having an addition of one nucleotide to any site of 5′-GAAAC-3′ (SEQ ID No: 21).

Examples of the above-described modified sequence may include 5′-NAAAC-3′ (SEQ ID No: 22), 5′-GNAAC-3′ (SEQ ID No: 23), 5′-GANAC-3′ (SEQ ID No: 24), 5′-GAANC-3′ (SEQ ID No: 25), and 5′-GAAAN-3′ (SEQ ID No: 26) (provided that N is A, T, G or C).

Examples of the PIPA according to the present embodiment may include the compounds represented by the following Formulae (I), (II), (III), and (IV):

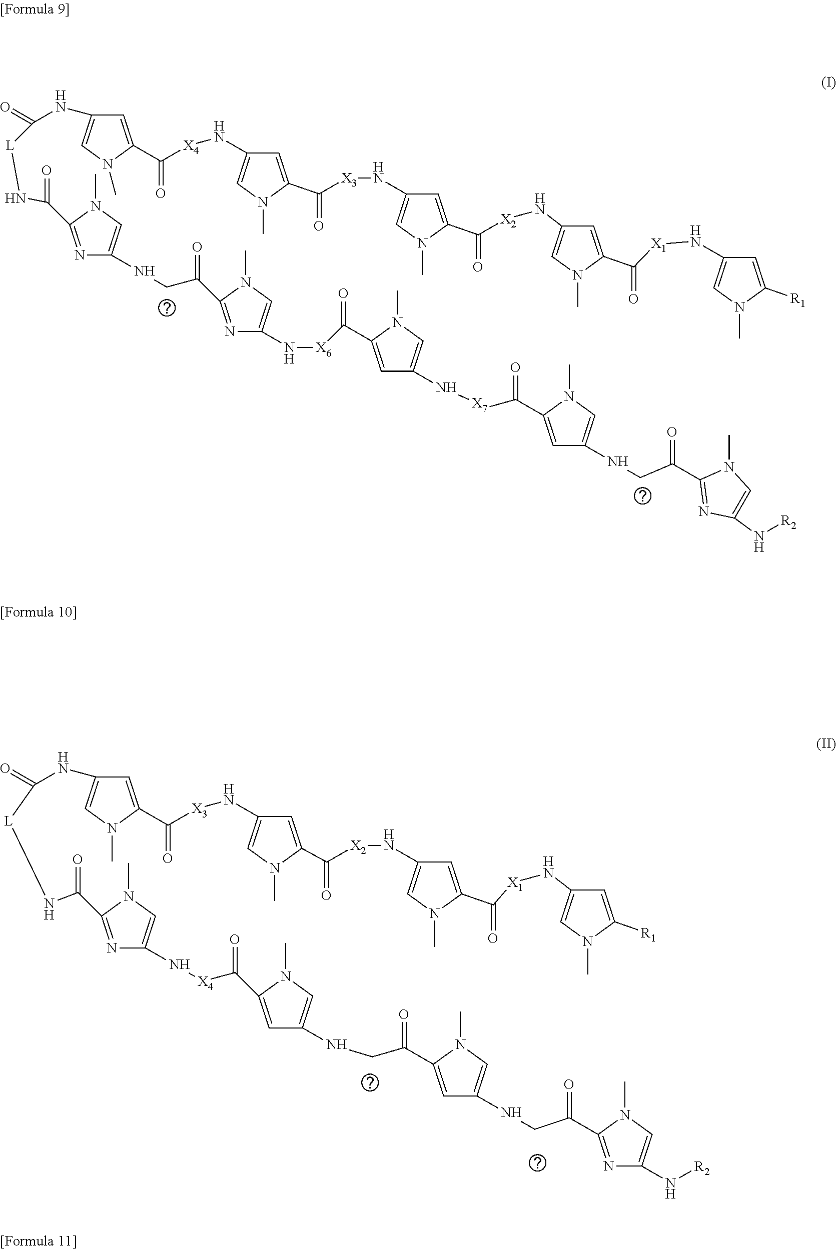

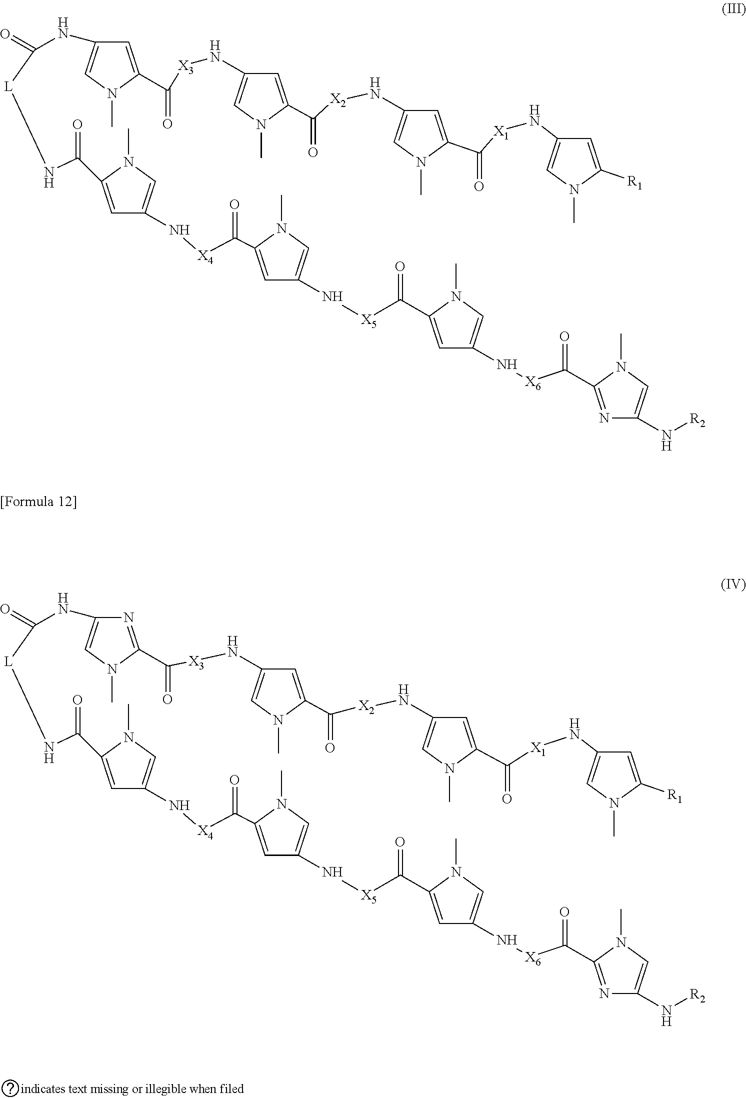

In the above Formulae (I), (II), (III), and (IV), L represents a C2 to 6 alkyl linker, and a γ-aminobutyric acid residue, etc. may be included therein, although it is not particularly limited thereto.

R₁ and R₂ each represent a C1 to C10 alkyl group optionally having a substituent or a C1 to C10 alkyl group optionally containing an amide bond and/or an amino group, or R₁ and R₂ may bind to each other to form a C2 to 6 alkyl linker.

X₁, X₂, X₃, X₄, X₅, X₆, X₇ and X₈ identically or independently represent a bond or an aliphatic amino acid residue. Examples of the aliphatic amino acid residue may include β-alanine, glycine, γ-aminobutyric acid and 5-aminovaleric acid, but are not particularly limited thereto.

Further specifically, examples of the PIPA according to the present embodiment may include the compounds represented by the following Formulae (Ia), (IIa), (IIIa), and (IVa):

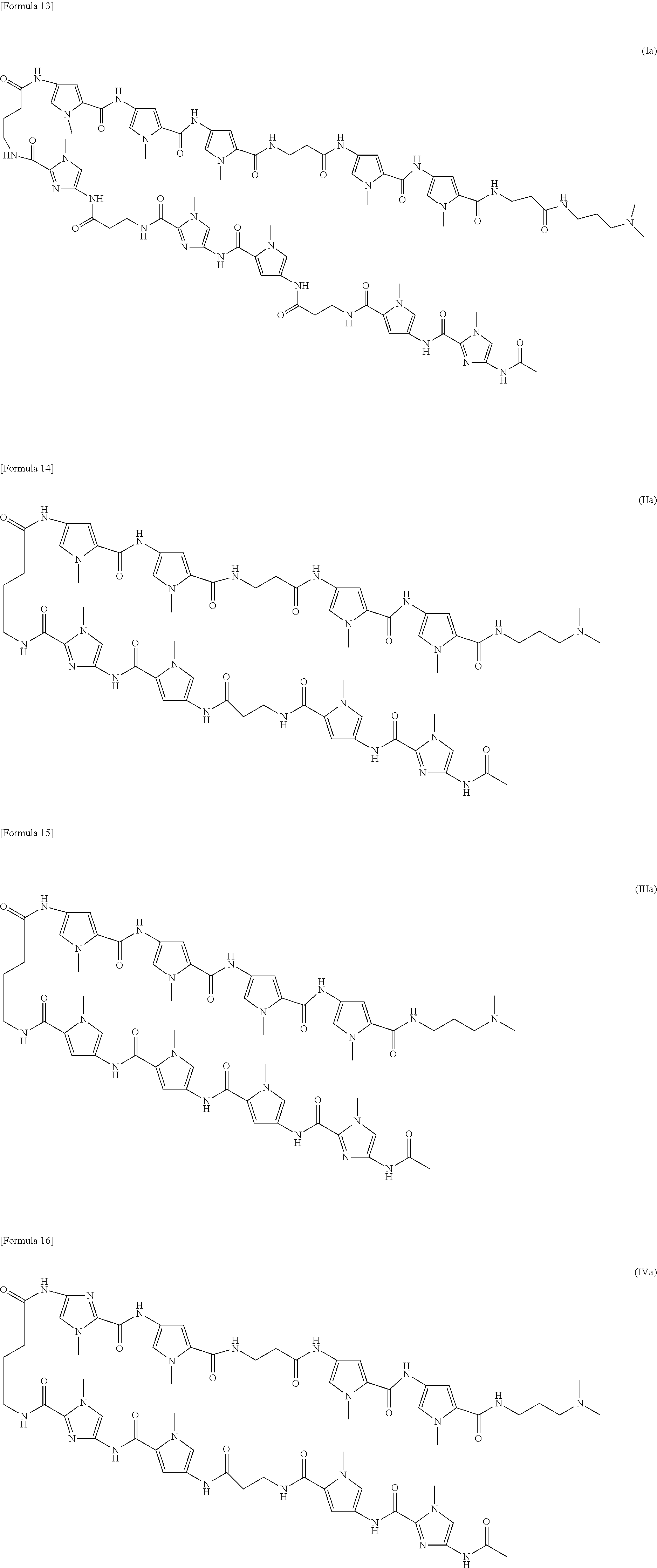

The PIPA according to the present embodiment can bind to the binding region with good affinity. When the affinity is expressed as a dissociation constant (Kd value), it is, for example, about 500 nM or less, about 400 nM or less, about 300 nM or less, about 200 nM or less, about 100 nM or less, about 90 nM or less, about 80 nM or less, about 70 nM or less, about 60 nM or less, about 50 nM or less, about 40 nM or less, about 30 nM or less, about 20 nM or less, or more preferably, about 10 nM or less, although the Kd value is not particularly limited thereto.

A second embodiment relates to: a PIPA for inhibiting the function of an IRF family transcription factor, wherein the PIPA specifically binds to the binding region of the IRF family transcription factor; use of the PIPA; or a composition comprising the PIPA.

Further, a third embodiment relates to an IRF family transcription factor inhibitor, comprising a PIPA that specifically binds to the binding region of the IRF family transcription factor (hereinafter also referred to as “the IRF transcription factor inhibitor according to the present embodiment”).

The PIPA in the second and third embodiments is the PIPA according to the present embodiment. The PIPA according to the present embodiment has a function of specifically binding to a binding sequence (e.g., an IRF-E sequence or an ISRE sequence) present in the promoter region of a target gene whose transcription is activated an IRF family transcription factor, with good affinity, so as to suppress or inhibit activation of the transcription of the target gene by the IRF family transcription factor. Known examples of the gene whose transcription is activated by the IRF family transcription factor, namely, the gene having the binding sequence of the IRF family transcription factor in the promoter region thereof, may include type I IFN (Interferon) (a generic name for 13 types of cytokines including IFN-μ, IFN-β, IFN-ε, IFN-κ, and IFN-ω) genes, and various genes such as an IL-6 gene and a TNF-α gene. Therefore, the IRF transcription factor inhibitor according to the present embodiment can be effectively used to suppress or inhibit transcription of genes whose activation of transcription is controlled by the IRF family.

It is to be noted that, in the present description, the “gene” means a nucleotide sequence or a nucleic acid that encodes genetic information. This nucleic acid includes polynucleotide, RNA, and DNA, and is not necessarily a nucleic acid that exists in a living organism, but it also includes a nucleic acid sequence that encodes any given protein (e.g., cDNA, etc.) and is artificially synthesized. In addition, the gene may be derived from any biological species.

A fourth embodiment relates to a medicament or a pharmaceutical composition, comprising, as an active ingredient, the IRF family transcription factor inhibitor according to the third embodiment or a PIPA conjugate according to a 11th embodiment (i.e. comprising, as an active ingredient, the PIPA or the PIPA conjugate according to the present embodiment) (hereinafter also referred to as “the medicament according to the present embodiment” or “the pharmaceutical composition according to the present embodiment,” or also, collective referred to as “the medicament, etc. according to the present embodiment”). Otherwise, the fourth embodiment relates to use of the PIPA according to the present embodiment in production of medicaments or pharmaceutical compositions.

PIPA is stably present in a living body without being degraded by peptidase, etc. In addition, since PIPA has a property of easily passing through the cell membrane, it is not necessary to use a means for introducing PIPA into a cell, such as DDS (drug delivery system). Thus, PIPA is a substance extremely excellent as an active ingredient for medicaments, etc. The PIPA according to the present embodiment suppresses or inhibits the activity of IRF family transcription factors, so that it can suppress the expression of various cytokine genes such as an IL-6 gene and a TNF-α gene, as well as type I IFN genes. Accordingly, PIPA exhibits effects as a medicament effective as a medicament for suppressing excessive immune response caused by abnormal expression of cytokines, or as a medicament for the prevention or treatment of diseases or infections provoked by excessive immune response. The medicament, etc. according to the present embodiment has the effect of suppressing excessive immune response, and can be used preventively or therapeutically. Examples of the disease provoked by excessive immune response may include, but are not particularly limited to: autoimmune diseases such as type 1 diabetes, rheumatoid arthritis, systemic lupus erythematosus (SLE), Goodpasture's syndrome, Graves' disease, Hashimoto's disease, multiple sclerosis, and autoimmune hemolytic anemia; viral infections, such as COVID19 that induces an excessive immune response, and influenza virus infection; and infections caused by pathogens such as bacteria, fungi, and protozoa.

With regard to the medicament according to the present embodiment, the active ingredient (the PIPA according to the present embodiment) itself may be administered. In general, however, the medicament according to the present embodiment is desirably administered in the form of a pharmaceutical composition comprising one or two or more pharmaceutical additives, as well as one or more substances serving as an active ingredient(s). In addition, the medicament, etc. according to the present embodiment may also comprise other components that are effective for suppressing excessive immune response.

The dosage form of the medicament, etc. according to the present embodiment may include, but are not particularly limited to, liquid preparations such as an injection and an infusion. The liquid preparation may be dissolved or suspended in water or other suitable solvent when used. When a liquid preparation is used as an injection or an infusion, it is prepared by dissolving the active ingredient in water, but may be dissolved in a normal saline or glucose solution if necessary, or buffers or preservatives may be added.

The types of pharmaceutical additives used in production of the medicament, etc. according to the present embodiment, the ratio of the pharmaceutical additives to the active ingredient, and a method for producing the medicament, etc. may be appropriately determined by a person skilled in the art, depending on the form of the medicament, etc. to be produced. As such pharmaceutical additives, inorganic or organic substances, or solid or liquid substances can be used. In general, such pharmaceutical additives can be added at a weight percentage of 1% to 90% based on the weight of the active ingredient. Specific examples of the pharmaceutical additives include lactose, glucose, mannit, dextrin, cyclodextrin, starch, sucrose, magnesium aluminometasilicate, synthetic aluminum silicate, carboxymethylcellulose sodium, hydroxypropyl starch, carboxymethylcellulose calcium, ion-exchange resin, methyl cellulose, gelatin, gum Arabic, hydroxypropyl cellulose, hydroxypropylmethyl cellulose, polyvinylpyrrolidone, polyvinyl alcohol, light anhydrous silicic acid, magnesium stearate, talc, tragacanth, bentonite, Veegum, titanium oxide, sorbitan fatty acid ester, sodium lauryl sulfate, glycerin, fatty acid glycerin ester, purified lanolin, glycerinated gelatin, polysorbate, macrogol, vegetable oil, wax, liquid paraffin, white petrolatum, fluorocarbon, nonionic surfactant, propylene glycol, and water.

In order to produce an injection, the active ingredient, together with a pH adjuster such as hydrochloric acid, sodium hydroxide, lactose, lactic acid, sodium, sodium monohydrogen phosphate or sodium dihydrogen phosphate, and an isotonizing agent such as sodium chloride or glucose, as necessary, is dissolved in a distilled water for injection, and the obtained solution is subjected to aseptic filtration and is then filled into an ampule. Otherwise, mannitol, dextrin, cyclodextrin, gelatin or the like may be further added to the above obtained solution, and the obtained mixture may be then subjected to vacuum freeze-drying, so as to prepare an injection which is dissolved before use. Furthermore, lecithin, polysorbate 80, polyoxyethylene hydrogenated castor oil or the like is added to the active ingredient, and it is emulsified in water, so as to prepare an emulsion for injection.

The dose, administration frequency, and administration method of the medicament, etc. according to the present embodiment are not particularly limited. These factors can be appropriately selected by a doctor's judgment, depending on conditions such as the purpose of preventing and/or treating deterioration and/or progression of a target disease to be prevented or treated, the type of the disease, and the body weight and age of a patient.

In general, when the medicament, etc. according to the present embodiment is used as an injection, the injection may be continuously or intermittently administered at a dose of 0.001 to 1000 mg (the weight of the active ingredient) per adult per day.

The medicament, etc. according to the present embodiment may be prepared as a sustained-release preparation such as an implantable tablet and a delivery system encapsulated into a microcapsule, using a carrier capable of preventing the sustained-release preparation from immediately being removed from the body. Such carriers that can be used herein include biodegradable and biocompatible polymers, such as ethylene vinyl acetate, polyanhydride, polyglycolic acid, collagen, polyorthoester, and polylactic acid. Such materials can be easily prepared by a person skilled in the art. In addition, a liposome suspension can also be used as a pharmaceutically acceptable carrier. The type of the liposome is not limited. Such liposome can be prepared as a lipid composition comprising phosphatidyl choline, cholesterol and PEG-induced phosphatidylethanol (PEG-PE) by being passed through a filter with a suitable pore size, such that it has a size suitable for use, and it can be then purified by a reverse phase evaporation method.

The medicament, etc. according to the present embodiment may be provided in the form of a kit, together with an instruction manual for administration method, etc. A drug contained in the kit is supplied with a container made of a material that effectively keeps the activity of the constituents of the therapeutic drug, etc. for a long period of time, does not adsorb the constituents on the inner surface thereof, and does not alter the quality of the constituents. For example, a sealed glass ampule may comprise a buffer enclosed in the presence of neutral non-reactive gas such as nitrogen gas.

Moreover, the kit may also comprise an instruction manual. The instruction manual for the present kit may be printed on a paper, etc., or may be stored on an electromagnetically readable medium, such as CD-ROM or DVD-ROM, and may be then provided to a user in such a form.

A fifth embodiment relates to an adjuvant comprising the PIPA according to the present embodiment (hereinafter also referred to as “the adjuvant according to the present embodiment”).

Recently, it has been reported that type I IFN induces inhibition of transcription of mRNA and degradation of mRNA, and reduces the effects of an mRNA vaccine (Linares-Fernandez et al., Trends Mol Med 26:311-323 doi: 10.1016/j.molmed.2019.10.002. 2020; De Beuckelaer et al., Mol Ther. 24:2012-2020, 2016.). Since the PIPA according to the present embodiment suppresses or inhibits the expression of type I IFN at a transcriptional level, it is expected that the effects of an mRNA vaccine is enhanced by mixing the PIPA into the mRNA vaccine preparation. That is to say, since it is considered that the PIPA according to the present embodiment suppresses the expression of type I IFN, and assists or enhances the action of mRNA (the expression of an antigen in a living body), the PIPA can also be used as an adjuvant or an adjuvant-like additive.

The adjuvant according to the present embodiment may comprise the PIPA according to the present embodiment in an amount of, for example, approximately 0.01% to 99.99% by weight. Moreover, the adjuvant according to the present embodiment may also comprise components that do not inhibit the function of the PIPA as an adjuvant, other than the PIPA according to the present embodiment. Examples of such components may include a stabilizer, a pH adjuster, a preservative, an antiseptic, and a buffer. Furthermore, the adjuvant according to the present embodiment may also comprise components that are known to be comprised in existing mRNA vaccine adjuvants and to have an immunostimulating activity, other than the PIPA according to the present embodiment.

The adjuvant according to the present embodiment can be used to all organisms having immune mechanisms.

A sixth embodiment relates to a vaccine comprising the adjuvant according to the present embodiment and mRNA (hereinafter also referred to as “the vaccine according to the present embodiment”).

The mRNA comprised in the vaccine according to the sixth embodiment is not particularly limited, as long as it expresses a protein that provokes immune responses. Examples of the mRNA may include mRNAs that express proteins, etc. derived from viruses, bacteria, parasites, fungi, rickettsia, chlamydia, prions, cancer cells, normal cells, etc.

A seventh embodiment relates to a method for preventing or treating excessive immune responses, or diseases and/or infections provoked by excessive immune responses, wherein the method comprises administering the medicament, etc. according to the present embodiment to patients.

Herein, the term “to treat” is used to mean to inhibit or to alleviate the progression and deterioration of pathologic conditions developed in a mammal affected with a disease as a therapeutic subject. On the other hand, the term “to prevent” is used to mean to inhibit in advance the onset of a disease as a preventive subject in a mammal that will be likely to be affected with the disease. The “mammal” as a preventive or therapeutic subject means any given animal classified into mammals, and thus, is not particularly limited. Examples of the mammal used herein may include: humans; pet animals such as dogs, cats, rabbits, ferrets, mice and hamsters; and livestock animals such as bovines, pigs, sheep and horses. The particularly preferable “mammal” is a human.

An eighth embodiment relates to a method for producing an IRF family transcription factor inhibitor comprising a PIPA, wherein
the method comprises a step of providing the binding region of an IRF family transcription factor, and a step of producing a PIPA specifically binding to the binding region.

In this method, the step of producing a PIPA that specifically binds to the binding region of an IRF family transcription factor may comprise a step of designing a polymer of pyrrole and/or imidazole that are selected to correspond to the nucleotide sequence existing in the binding region, and as necessary, designing a polymer, in which an aliphatic amino acid residue such as β-alanine or γ-aminobutyric acid is inserted into the polymer of pyrrole and/or imidazole, and then actually synthesizing the polymer.

A ninth embodiment relates to a PIPA that specifically binds to the binding region of an IRF family transcription factor, which is for use as a pseudo-transcription factor, or a composition comprising a PIPA specifically binding to the binding region of an IRF family transcription factor, which is for use as a pseudo-transcription factor.

As mentioned above, the PIPA according to the present embodiment has the property of binding to a genomic DNA sequence, to which an IRF family transcription factor specifically binds, and the present PIPA can inhibit the transcription performed by the IRF family transcription factor via the binding sequence, and/or the activation thereof. Accordingly, the PIPA according to the present embodiment has the function of a substance that inhibits the transcription performed by the IRF family transcription factor via the binding sequence, and or the activation thereof, namely, the function of a “pseudo-transcription factor.” Therefore, the PIPA according to the present embodiment can be used also as a pseudo-transcription factor. For the PIPA, please refer to the explanations in other embodiments.

A tenth embodiment relates to a method of using the PIPA as a pseudo-transcription factor in a subject, wherein the method comprises a step of applying an effective amount of the PIPA that specifically binds to the binding region of the IRF family transcription factor to the subject.

In this method, the “subject” means any given animal classified into mammals, and thus, is not particularly limited. Examples of the subject used herein may include: humans; pet animals such as dogs, cats, rabbits, ferrets, mice and hamsters; and livestock animals such as bovines, pigs, sheep and horses. The particularly preferable “mammal” is a human. For the PIPA, please refer to the explanations in other embodiments.

An eleventh embodiment relates to a conjugate comprising the PIPA according to the present embodiment (i.e. the PIPA that specifically binds to the binding region of the IRF family transcription factor) and a PIPA auxiliary factor (hereinafter also referred to as “the PIPA conjugate according to the present embodiment”). The “PIPA auxiliary factor” is used in the present embodiment to mean a substance different from the PIPA, which enhances the function of the PIPA according to the present embodiment. The PIPA according to the present embodiment has the function of suppressing excessive immune response through suppressing the expression of a gene whose transcription is activated by the IRF family transcription factor. Accordingly, the “PIPA auxiliary factor” in the present embodiment includes a substance that enhances the function of the PIPA (e.g., an immune response-suppressing function). Further, the “PIPA auxiliary factor” in the present embodiment also includes substances that assist the action of the PIPA, such as, for example, a substance that improves the stability of the PIPA in vivo, a substance that enhances the transport of the PIPA to target organs or target cells, and a substance that enhances introduction of the PIPA into cells, but examples of the PIPA auxiliary factor are not limited thereto.

The PIPA auxiliary factor of the present embodiment can be any substance, as long as it binds to the PIPA and does not inhibit the intracellular delivery of the PIPA. As such PIPA auxiliary factors, for example, those having a low molecular weight (about 500 to about 2,000) are preferable, and those having a molecular weight that does not affect the introduction efficiency of the PIPA into cells, for example, those having a molecular weight of about 500 to about 1,000, are further preferable. Such a PIPA auxiliary factor forms a conjugate with the PIPA according to the present embodiment, so that it enhances the function of the PIPA and/or assists the action of the PIPA. Accordingly, the PIPA auxiliary factor of the present embodiment exhibits preventive effects and/or therapeutic effects on, for example, diseases or infections that are provoked by excessive immune response.

The substance that enhances the function of the PIPA according to the present embodiment is not particularly limited, and examples thereof may include immunosuppressive substances such as hydroxychloroquine and FK506, and anti-inflammatory substances such as various steroids. On the other hand, the substance that assists the action of the PIPA of the present embodiment may include vitamins (e.g., vitamin E and vitamin A), lipids such as cholesterol, and anisamide.

The PIPA and the PIPA auxiliary factor according to the present embodiment may directly bind to each other to form a conjugate, or may also be linked to each other via a linker. As such a linker, any given linker can be utilized, as long as the PIPA conjugate according to the present embodiment can exhibit its function, for example, the PIPA conjugate can exhibit the function of a drug delivery system (DDS), and thus, the linker is not particularly limited. The linker may be, for example, an alkyl linker containing 1 to 6 carbon atoms (C1 to C6), etc. The linker may comprise a functional group for binding the PIPA according to the present embodiment with the PIPA auxiliary factor, such as, for example, a maleimide group, a thiol group, an amino group, a carboxyl group, a hydroxyl group, or an azide group. The site of the PIPA, to which the PIPA auxiliary factor is allowed to bind, is not particularly limited, and it may be, for example, the terminal portion of the PIPA (e.g., R₁ or R₂, etc. in Formulae (I), (II), and (III)), etc.

When the present description is translated into English and the English description includes singular terms with the articles “a,” “an,” and “the,” these terms include not only single items but also multiple items, unless otherwise clearly specified from the context that it is not the case. In addition, in the present descriptions, the term “about” or “approximately” is used to mean a numerical range of ±10%.

Hereinafter, the present invention will be further described in the following examples. However, these examples are only illustrative examples of the embodiments of the present invention, and thus, are not intended to limit the scope of the present invention.

### EXAMPLES

### 1. Materials and Methods

### 1-1. Cells

Raw264.7 cells, a cell line, were purchased from RIKEN Cell Bank, and were then cultured using D-MEM (Nacalai Tesque) containing 10% FBS (SIGMA-ALDRICH) and Penicillin-Streptomycin mixed solution (Nacalai Tesque).

Mouse peritoneal macrophages were harvested from 6-week-old CL57B/6 mice (CLEA Japan, Inc.) by peritoneal lavage using 7 mL of PBS (Nacalai Tesque), and the cells adhered to the culture plate by culture for 2 hours were used in experiments. The culture was carried out using an RPMI or D-MEM medium (Nacalai Tesque) containing 10% FBS (SIGMA-ALDRICH) and Penicillin-Streptomycin mixed solution (Nacalai Tesque). The CL57B/6 mice were bred in an SPF environment.

### 1-2. Determination of IRF Binding Sequence and Synthesis of PIPA

The IRF family transcription factor binds to the consensus sequence shown in 5′-AANNGAAA-3′ (SEQ ID No: 27). Thus, based on this consensus sequence, 5′-GAAAGTG-3′ (SEQ ID No: 1), 5′-GAAAG-3′ (SEQ ID No: 9), 5′-GAAAA-3′ (SEQ ID No: 15) and 5′-GAAAC-3′ (SEQ ID No: 21) were selected as binding sequences for designing a PIPA, and then, the synthesis of a PIPA that binds to these sequences was entrusted to PEPTIDE INSTITUTE, INC.

### 1-3. Measurement of Cytokine Concentration

2×10⁴ Raw264.7 cells were seeded on a plate, and were then treated with IRF-PIPA 1, IRF-PIPA 2, IRF-PIPA 3 or IRF-PIPA 4 for 24 hours. Thereafter, the cells were stimulated with LPS (Lipopolysaccharide, SIGMA-ALDRICH) for 16 hours, and the culture supernatant was then recovered. The concentration of cytokine in the culture supernatant was measured using ELISA kit (R&D SYSTEMS) according to the standard protocols. The culture supernatant was diluted with 1% BSA-PBS, as necessary, and was then analyzed. 2.5×10⁵ Mouse peritoneal macrophages were seeded on a plate, and were then treated with IRF-PIPA 3 for 16 or 24 hours. Thereafter, the cells were stimulated with Poly(I:C) (InvivoGen) for 16 hours or with mRNA vaccine (Moderna) for 20 to 24 hours, and the culture supernatant was then recovered. The concentration of cytokine in the culture supernatant was measured using ELISA kit (R&D SYSTEMS) according to the same standard protocols. On the other hand, the concentration of cytokine in mouse plasma was measured by administering 400 μg/mouse IRF-PIPA 3 into the abdominal cavity of wild-type mice, then administering 250 μg of Poly(I:C) into the same abdominal cavity 24 hours after the IRF-PIPA 3 administration, then collecting blood further 2 hours after the Poly(I:C) administration, and then measuring the cytokine concentration in the plasma using ELISA kit (R&D SYSTEMS) according to standard protocols.

1-4. Measurement of mRNA Level

2.5×10⁵ Peritoneal macrophages were seeded on a plate, and were then treated with IRF-PIPA 3 for 24 hours. The cells were recovered 2, 4, or 6 hours after they had been stimulated with Poly(I:C) (InvivoGen), or 20 hours after they had been stimulated with mRNA vaccine (Moderna). Thereafter, RNA was extracted from the cells, using TRIsure (Nippon Genetics Co., Ltd.) and RNA Extraction Kit (ZYMO RESEARCH) according to standard protocols. After that, cDNA was prepared from the extracted RNA using 5× PrimeScrip^{tRT} Master Mix (Takara Bio, Inc.), and qPCR was then carried out using Sybr green (BioLabs) and primers specific to individual genes (FASMAC). Light Cycler480 (Roche, Switzerland) was used for the qPCR. As primer sequences, general sequences used in the previous references (Proc Natl Acad Sci U.S.A. 2019 Nov. 19; 116 (47): 23653-23661. doi: 10.1073/pnas. 1915326116), etc. were employed.

1-5. Measurement of dsDNA Concentration and LDH Activity

The concentration of dsDNA in the culture supernatant was measured using Nano drop one (Thermo Fisher SCIENTIFIC). In LDH assay, LDH activity was measured using Cytotoxicity LDH Assay Kit-WST (DOJINDO LABORATORIES) according to standard protocols. As a positive control, a lysate obtained by mixing 2.5×10⁵ peritoneal macrophages with 90 μL of 10% FBS-RPMI or D-MEM medium and 10 μL of Lysis Buffer, and then disrupting the cells, was used.

### 1-6. Measurement of Concentration of SARS-COV-2 Spike Protein Receptor Binding Domain (RBD)

IRF-PIPA 3 was administered in an amount of 400 μg/mouse into the muscle (i.m.) or abdominal cavity (i.p.) of wild-type mice. Twenty-four hours after the administration, mRNA vaccine against SARS-COV-2, manufactured by Moderna, was administered in an amount of 1 μg/mouse into the muscle, and at the same time, 250 μg/mouse Poly(I:C) was administered into the abdominal cavity or the muscle. One day later, blood was collected from the mice, and the RBD concentration in the plasma was measured by an ELISA method.

### 1-7. Analysis of Intermolecular Interaction

The analysis was performed using Octet K2 (ForteBio) according to standard protocols. A streptavidin-conjugated sensor (ForteBio) was used, and a biotin-labeled target sequence (FASMAC) was allowed to bind to the sensor. Further, according to standard protocols, the binding of 10 to 320 nM IRF-PIPA 3 and the target sequence, and dissociation thereof were analyzed for 60 seconds each. From these results, a Kd value was calculated using Data Analysis (Sartorius Japan K.K.).

### 2. Results

2-1. Acquisition of PIPA that Suppresses Type I IFN Induction

Although IRF transcription factor-binding sequences have been reported by in vitro analysis, there are no reports regarding the analysis of the physiological importance of these sequences, using knock-in mice of the promoter region, etc. Thus, studies have been first conducted regarding which target sequence of PIPA, on which the PIPA exhibits strong inhibitory effects. The IRF transcription factor-binding sequences predicted from previous reports were narrowed down to 4 sequences, namely, 5′-GAAAGTG-3′ (SEQ ID No: 1), 5′-GAAAG-3′ (SEQ ID No: 9), 5′-GAAAA-3′ (SEQ ID No: 15) and 5′-GAAAC-3′ (SEQ ID No: 21), and a PIPA binding to each of the sequences was then prepared. Suppression of IFN-β whose expression is induced depending on the IRF transcription factor was verified using Raw264.7 cells as a macrophage cell line. The structures of PIPAs binding to 5′-GAAAGTG-3′ (SEQ ID No: 1), 5′-GAAAG-3′ (SEQ ID No: 9), 5′-GAAAA-3′ (SEQ ID No: 15) and 5′-GAAAC-3′ (SEQ ID No: 21) were represented by the following Formula (Ia), Formula (IIa), Formula (IIIa) and Formula (IVa), respectively. It is to be noted that, in the present description, the PIPAs represented by Formula (Ia), Formula (IIa), Formula (IIIa) and Formula (IVa) are also denoted as IRF-PIPA 3, IRF-PIPA 1, IRF-PIPA 2 and IRF-PIPA 4, respectively.

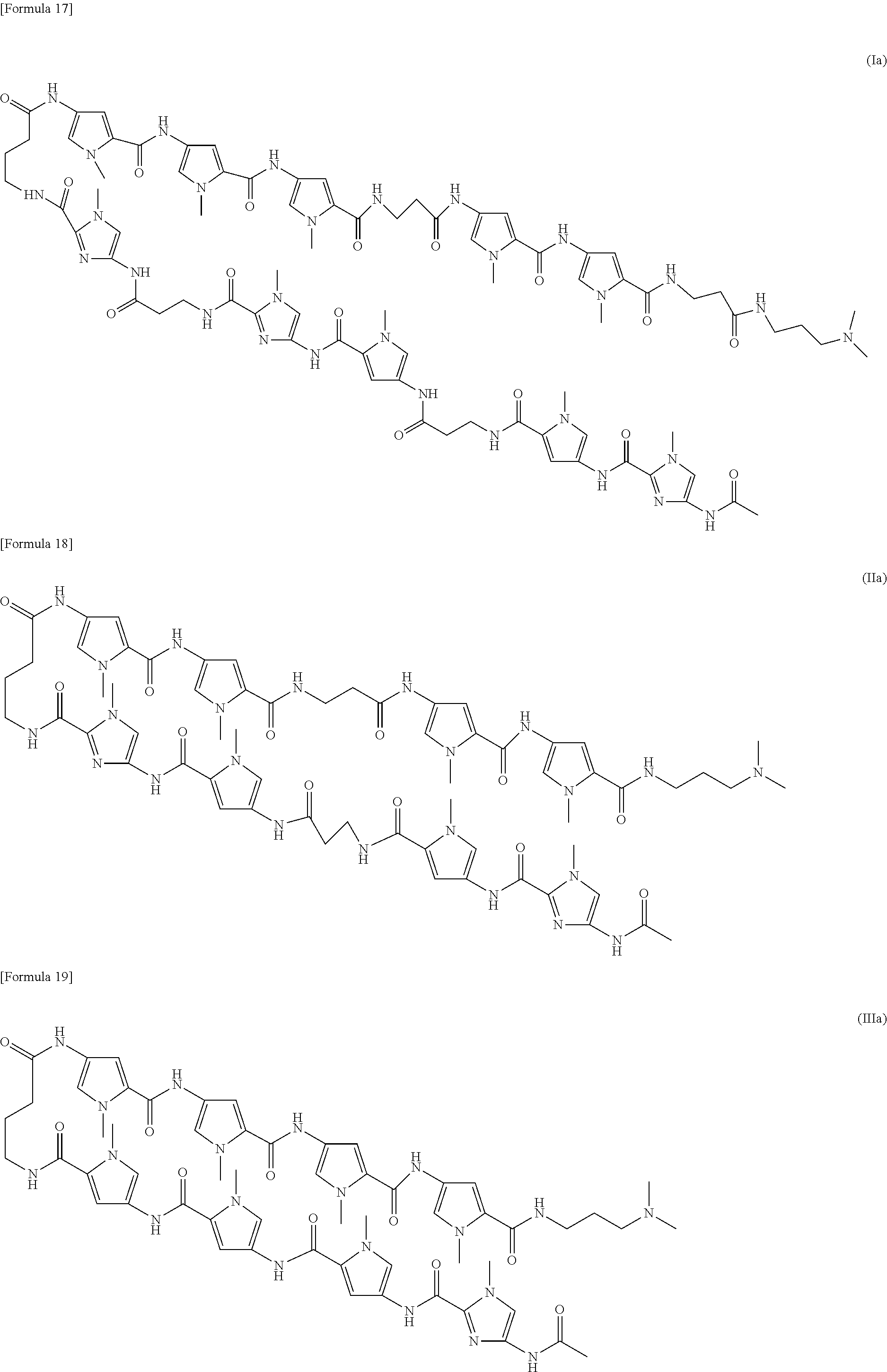

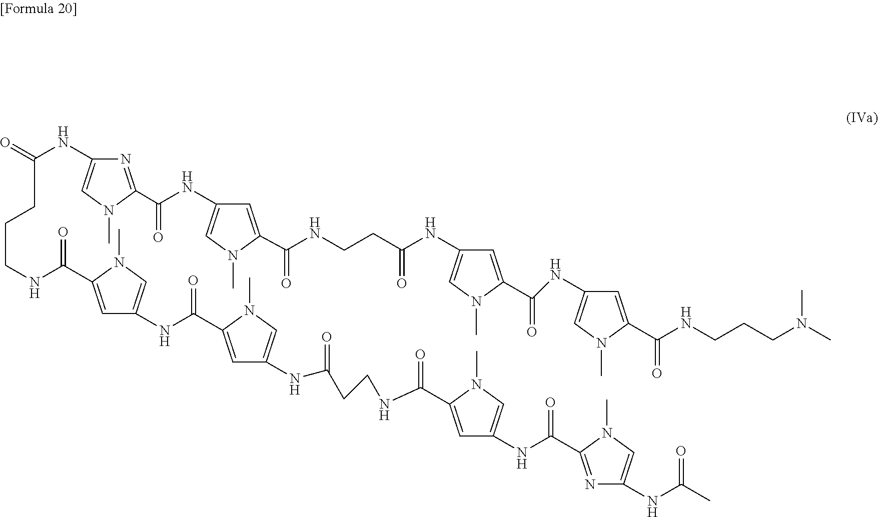 [SMILES: CC(=O)Nc1cn(C)c(C(=O)Nc2cc(C(=O)NCCC(=O)Nc3cc(C(=O)Nc4cc(C(=O)NCCCC(=O)Nc5cn(C)c(C(=O)Nc6cc(C(=O)NCCC(=O)Nc7cc(C(=O)Nc8cc(C(=O)NCCCN(C)C)n(C)c8)n(C)c7)n(C)c6)n5)n(C)c4)n(C)c3)n(C)c2)n1]

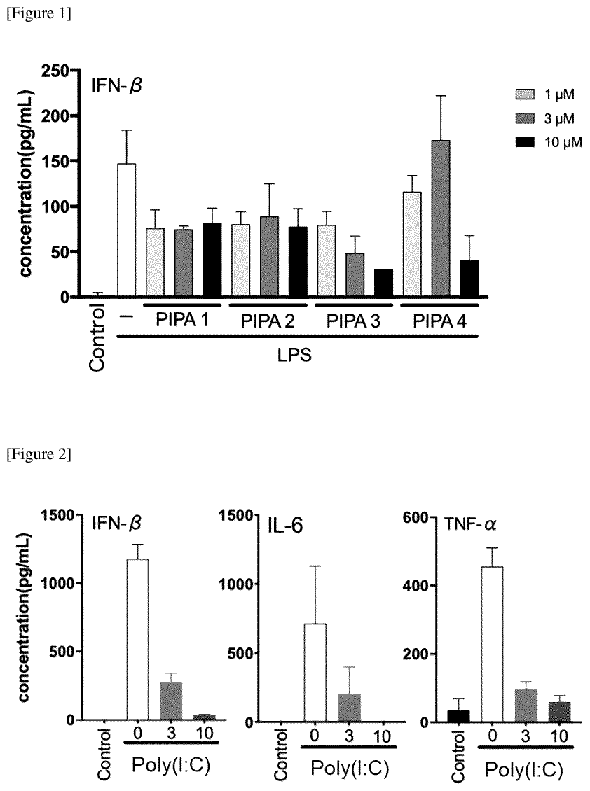

Raw264.7 cells as a macrophage cell line were treated with IRF-PIPAs 1 to 4 in advance, and were then stimulated with pathogen components inducing IFN-β production, and thereafter, the concentration of IFN-β in the culture supernatant was measured by ELISA. As a result, the suppressive effects of the PIPAs against IFN-β production could be confirmed in all of PIPAs 1 to 4 (FIG. 1). While concentration dependence was not observed for IRF-PIPA 1, IRF-PIPA 2 and IRF-PIPA 4, concentration-dependent suppression was observed for IRF-PIPA 3, which further showed the strongest suppressive action among the 4 PIPAs. In other words, since IRF-PIPA 3 was considered to most strongly suppress IFN-β production by the expected mechanism of transcriptional inhibition, verification was carried out using IRF-PIPA 3 in the following experiments.

### 2-2. Verification of Suppressive Ability of IRF-PIPA 3 Against Type I IFN Induction

Next, peritoneal macrophages were collected from mice, and the suppressive ability of IRF-PIPA 3 against IFN-b production was examined. After peritoneal macrophages had previously been treated with IRF-PIPA 3, the peritoneal macrophages were stimulated with double-stranded RNA (Poly(I:C)) as a pathogen component inducing IFN-b production, and the cytokine concentration in the culture supernatant was then measured by ELISA (FIG. 2). As a result, it was confirmed that IRF-PIPA 3 strongly suppresses IFN-β production in a concentration-dependent manner even in peritoneal macrophages. Further, IL-6 and TNF-α, which are cytokines known to be dependent on IRF transcription factors as with IFN-β, were also suppressed, although less strongly than IFN-β (FIG. 2). These results suggest that IRF-PIPA 3 broadly suppresses IRF transcription factor-dependent responses.

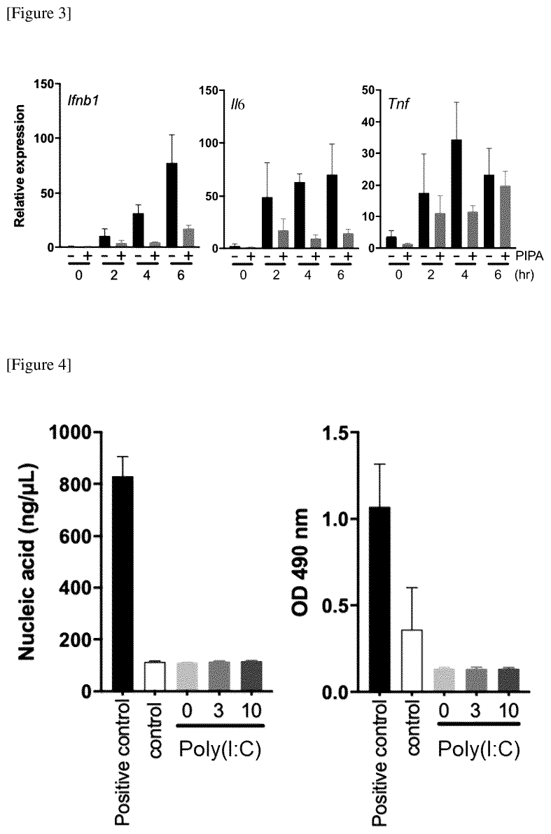

Furthermore, in order to verify whether IRF-PIPA 3 suppresses IFN-β production by the assumed mechanism, the same experiment as that mentioned above was carried out using peritoneal macrophages, and the relative expression level of mRNA was analyzed using a qRT-PCR method to evaluate the influence on transcription (FIG. 3). As a result, it was confirmed that IRF-PIPA 3 strongly suppressed induction of the expression of Ifnb1 mRNA in a concentration-dependent manner. Further, similar repression was observed for Il6 and Tnf mRNAs. These results suggest that IRF-PIPA 3 inhibits IRF transcription factor-dependent transcription.

### 2-3. Verification of Cytotoxicity of IRF-PIPA 3

In the mRNA analysis, the mRNA expression level of mRNA of interest is calculated as a relative value to the mRNA expression level of a gene that is not normally fluctuated (in the present example, glyceraldehyde-3-phosphate dehydrogenase: GAPDH), the mRNA level of a gene of interest can be verified without being affected by cell death or other factors. Thus, the results of the mRNA analysis shown in FIG. 3 strongly show that IRF-PIPA 3 has a suppressive ability. Meanwhile, from these results, it is not possible to evaluate whether cell death occurs in addition to the suppressive effects. In the first place, since the transcription factors bind to target sequences that exist in an infinite number of unimportant regions, even though they target specific sequences. Thus, it is considered that the same occurs for PIPA. In other words, it is suggested that PIPA binds to an infinite number of target sequences existing in the genome, and repeatedly performs a large number of non-significant bindings, behind inhibition of IRF transcription factors. From this point of view, it is extremely important to verify the on-target side effects of PIPA, and verification of its cytotoxicity as an output thereof is considered to have great importance.

Therefore, next, the cytotoxic activity of IRF-PIPA 3 was verified using peritoneal macrophages. The same experiment as that shown in FIG. 2 was carried out, and the release of DNA and a lactate dehydrogenase (LDH) protein from dead cells to the culture supernatant was analyzed as indicators of cytotoxicity. As a result, the amount of DNA in the culture supernatant was not increased at all by PIPA treatment (FIG. 4, left). Furthermore, in order to verify the release of LDH, LDH activity in the culture supernatant was measured. As a result, similarly, the LDH activity was not increased at all by PIPA treatment (FIG. 4, right). These results suggest that IRF-PIPA 3 does not exhibit cytotoxic activity, at least, in a concentration range in which IRF-PIPA 3 shows strong suppressive action.

### 2-4. Verification of Binding of IRF-PIPA 3 and Target Sequence

From the aforementioned results, it is suggested that PIPA 3 binds to the target sequence on the promoter of the target gene of the IRF transcription factor and suppresses the transcription thereof. Moreover, in order to confirm that IRF-PIPA 3 actually binds to a DNA sequence of interest, the binding of PIPA 3 and the target DNA sequence was verified in vitro. For the verification, Octet K2, which is capable of verifying the interaction between two molecules, was used, and the binding of IRF-PIPA 3 to the DNA sequence (GAAAGTG C GAAAGTG C GAAAGTG C: SEQ ID No: 28) immobilized on the sensor was detected. As a result, signals indicating the binding of IRF-PIPA 3 and the target sequence were detected in a concentration-dependent manner, and from such signals, the strength of the binding force was found to be 2.27×10⁻⁸ as a Kd value (FIG. 5). From these results, it was demonstrated that IRF-PIPA 3 certainly has an action to bind to the target sequence, which has been assumed.

### 2-5. Verification of Medicinal Effects of IRF-PIPA 3 at Living Body Level

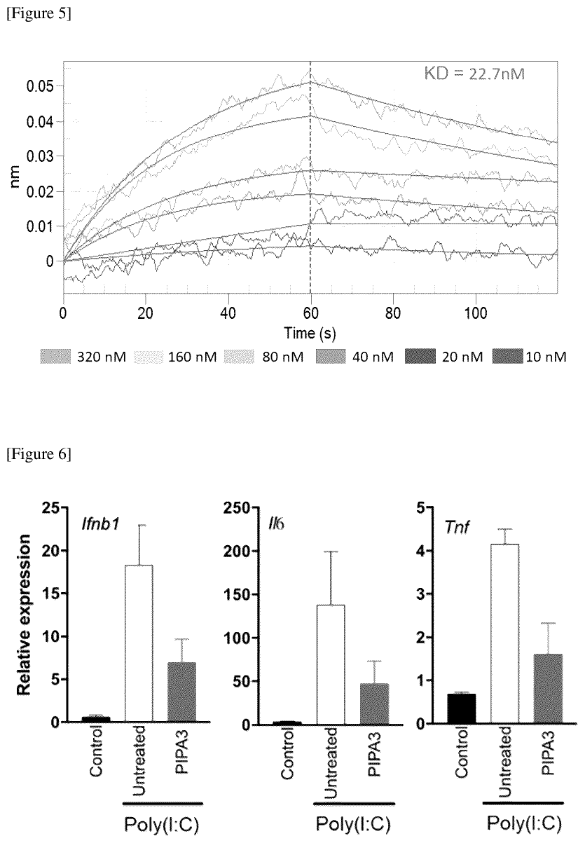

The transcription-suppressing effect of IRF-PIPA 3 in vivo was verified. IRF-PIPA 3 had previously been administered into the abdominal cavity of mice, and 3 hours after the administration, poly(I: C) was also administered as a pathogen component into the abdominal cavity of mice. Further, 2 hours after the poly(I: C) administration, peritoneal macrophages were collected from the mice, and the relative expression level of mRNA was analyzed using a qRT-PCR method (FIG. 6). Consequently, the obtained results were consistent with the in vitro results, IRF-PIPA 3 significantly suppressed the expression levels of Ifnb1, Il6 and Tnf mRNAs. From these results, it was demonstrated that IRF-PIPA 3 has the suppressive effect even in vivo, at least, at local positions.

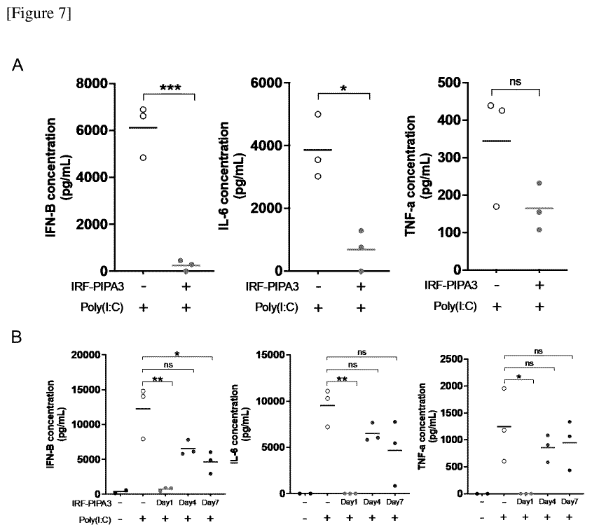

Next, IRF-PIPA 3 had previously been administered into the abdominal cavity of mice, and 24 hours, 1 day, 4 days or 7 days after the administration, poly(I: C) was also administered as a pathogen component into the abdominal cavity of mice. Further, 2 hours after the poly(I: C) administration, blood was collected from the mice, and the protein concentrations of IFN-β, IL-6 and TNF-α in the plasma were measured by an ELISA method. The results are shown in FIG. 7A (24 hours after IRF-PIPA 3 administration) and FIG. 7B (1 day, 4 days, or 7 days after IRF-PIPA 3 administration). IRF-PIPA 3 significantly suppressed the expression of individual proteins, IFN-β, IL-6 and TNF-α. The highest suppressive effect was observed 1 day after the IRF-PIPA 3 administration, and the effect was sustained until 7 days after the IRF-PIPA 3 administration, although the suppressive effect was attenuated. From these results, it was revealed that IRF-PIPA 3 effectively suppresses the expression of IRF family proteins in the living body as a whole.

2-6. Verification of Influence of IRF-PIPA 3 on Immune Response Caused by mRNA Vaccine

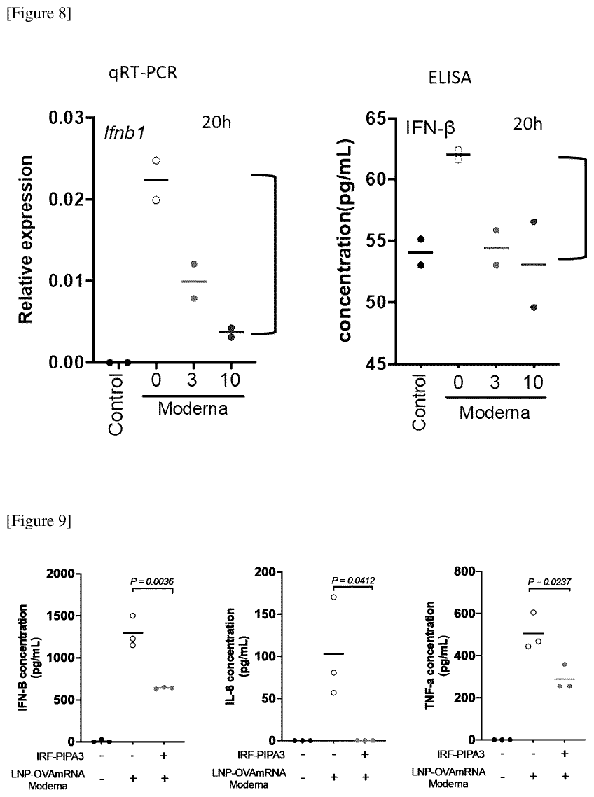

As mentioned above, it has been reported that type I IFN induces inhibition of mRNA transcription and degradation of mRNA, thereby reducing the effects of an mRNA vaccine. Thus, whether or not IFR-PIPA 3 can suppress IFN-β transcription after administration of the mRNA vaccine against SARS-COV-2, manufactured by Moderna, was examined. After peritoneal macrophages had previously treated with IRF-PIPA 3 (3 μM or 10 μM), they were treated with the mRNA vaccine (Moderna) and were then cultured for 20 hours. After completion of the culture, RNA was prepared from the recovered cells using TRIsure, and the relative expression level of the Ifnb1 gene was then measured by qRT-PCR (FIG. 8, left). In addition, after completion of the culture, the concentration of cytokine in the culture supernatant was measured by ELISA (FIG. 8, right). As a result, it was confirmed that IRF-PIPA 3 strongly suppressed IFN-b transcription after addition of the mRNA vaccine in a concentration-dependent manner in peritoneal macrophages.

Next, after peritoneal macrophages had previously treated with IRF-PIPA 3 (6 μM), they were treated with an mRNA vaccine (LNP-mRNA), in which an mRNA encoding Ovalbumin (OVA) was included in a Moderna-type lipid nano particle (LNP), instead of the mRNA vaccine against SARS-COV-2 manufactured by Moderna, and they were then cultured for 20 to 24 hours. After completion of the culture, the concentration of cytokine in the culture supernatant was measured by ELISA (FIG. 9). As a result, it was confirmed that IRF-PIPA 3 strongly suppressed the expression of individual proteins, IFN-β, IL-6 and TNF-α, in peritoneal macrophages in a concentration-dependent manner after addition of the mRNA vaccine.

By the way, it has been known that double-stranded RNA is generated as a foreign matter in the production process of LNP-mRNA (Linares-Fernandez et al., 2020 March Trends Mol Med 26:311-323 doi: 10.1016/j.molmed.2019.10.002.). Since double-stranded RNA induces the expression of type I IFNs, it is considered that the double-stranded RNA may reduce the effects of the vaccine. Thus, whether or not PIPA can ameliorate such a reduction in the effects of the mRNA vaccine due to the mixing of the double-stranded RNA as a foreign matter.

IRF-PIPA 3 was administered in an amount of 400 μg/mouse into the muscle (i.m.) or abdominal cavity (i.p.) of wild-type mice. Twenty-four hours after the administration, the mRNA vaccine against SARS-COV-2 manufactured by Moderna was administered in an amount of 1 μg/mouse into the muscle, and at the same time, 250 μg/mouse Poly(I: C) that was double-stranded RNA was administered as a foreign matter into the abdominal cavity or the muscle. One day after the administration, the blood was collected from the mice, and the concentration of RBD (Receptor Binding Domain; the spike protein receptor binding region of SARS-COV-2) in the plasma was measured by an ELISA method.

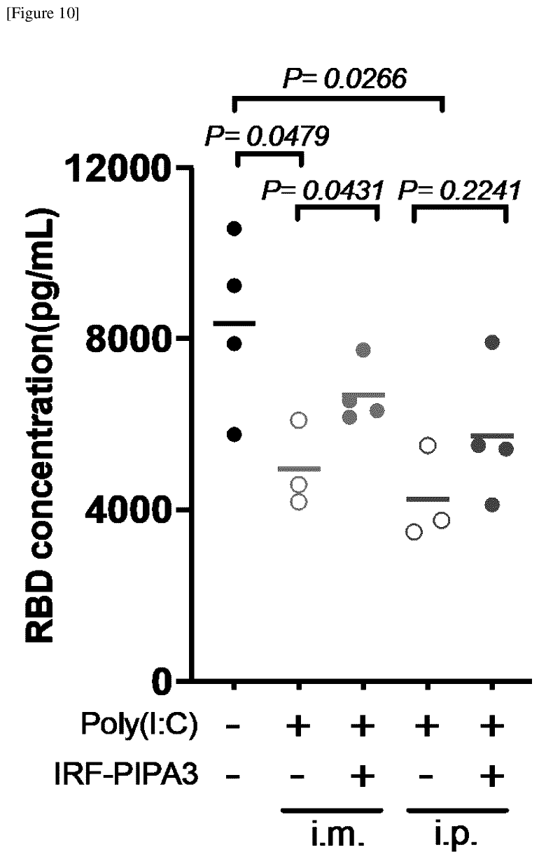

In the absence of IRF-PIPA 3, when Poly(I: C) was added as a foreign matter upon administration of the mRNA vaccine, the concentration of the RBD protein in the blood was decreased. In contrast, in the presence of IRF-PIPA 3, a decrease in the concentration of the RBD protein caused by Poly(I: C) was suppressed (FIG. 10).

The aforementioned results show that IRF-PIPA 3 suppresses transcription of IFN-β after administration of the mRNA vaccine, suggesting that IRF-PIPA 3 has a function as an adjuvant that assists or enhances the effects of the mRNA vaccine.

### INDUSTRIAL APPLICABILITY

The present invention provides a PIPA that suppresses the expression of cytokines (e.g., type I IFN, IL-6, TNF-α, etc.), which are essential for activation of immune response, at a transcription stage. Therefore, the present invention is expected to be utilized in the pharmaceutical and medical fields.

1. A pyrrole imidazole polyamide (PIPA), which specifically binds to the binding region of an IRF (IFN regulatory factor) family transcription factor.

2. The PIPA according to claim 1, which functions as a pseudo-transcription factor.

3. The PIPA according to claim 1, which has a binding affinity of about 500 nM or less as a dissociation constant (Kd value) to the binding region.

4. The PIPA according to claim 3,
wherein the binding region is a region comprising 5′-GAAAGTG-3′ or a modified sequence thereof, and
wherein said modified sequence comprises (a) 5′-GAAAGTG-3′, except that any one nucleotide is deleted or mutated, or (b) 5′-GAAAGTG-3′, except that one nucleotide is added to any site thereof.

5. The PIPA according to claim 3,
wherein the binding region is a region comprising 5′-GAAAG-3′ or a modified sequence thereof, and
wherein said modified sequence comprises (a) 5′-GAAAG-3′, except that any one nucleotide is deleted or mutated, or (b) 5′-GAAAG-3′, except that one nucleotide is added to any site thereof.

6. The PIPA according to claim 3,
wherein the binding region is a region comprising 5′-GAAAA-3′ or a modified sequence thereof, and
wherein said modified sequence comprises (a) 5′-GAAAA-3′, except that any one nucleotide is deleted or mutated, or (b) 5′-GAAAA-3′, except that one nucleotide is added to any site thereof.

7. The PIPA according to claim 3,
wherein the binding region is a region comprising 5′-GAAAC-3′ or a modified sequence thereof, and
wherein said modified sequence comprises (a) 5′-GAAAC-3′, except that any one nucleotide is deleted or mutated, or (b) 5′-GAAAC-3′, except that one nucleotide is added to any site thereof.

8. The PIPA according to claim 3, which is represented by the following Formula (I), (II), (III) or (IV): 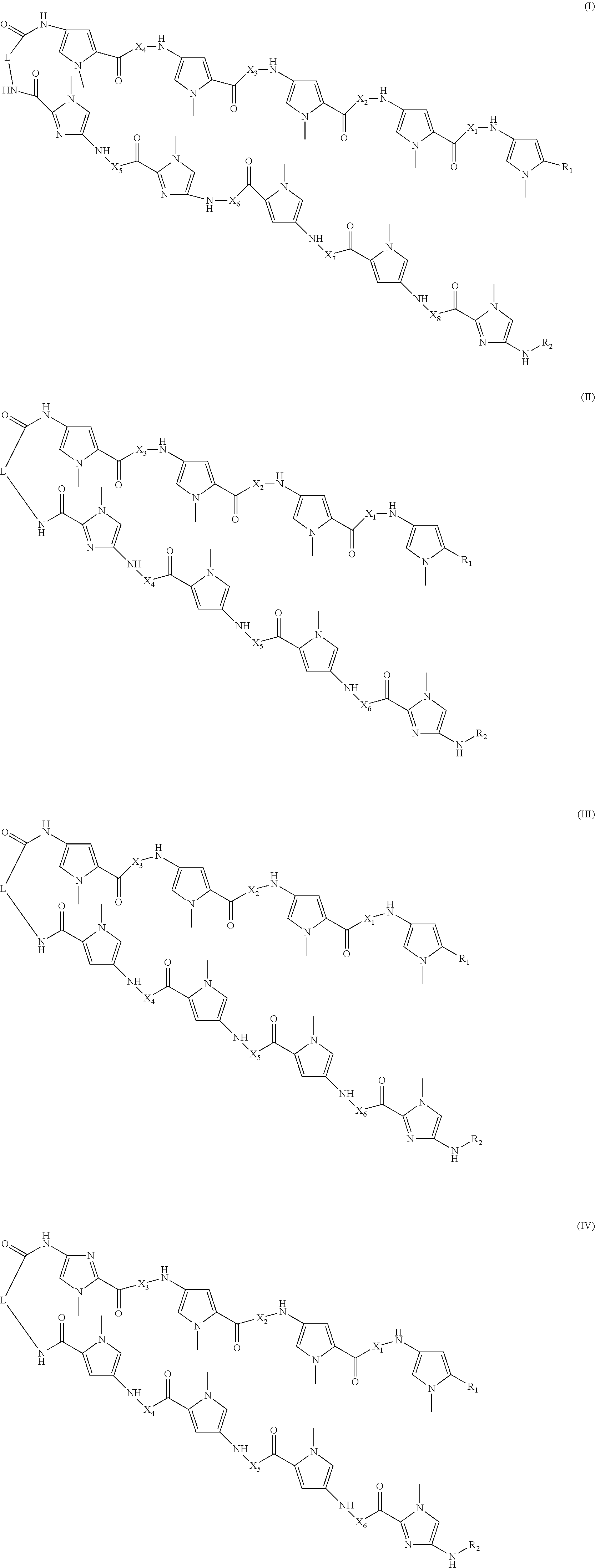
wherein in the Formulae (I), (II), (III) and (IV), L represents a C2 to 6 alkyl linker; R₁ and R₂ each represent a C1 to C10 alkyl group optionally having a substituent or a C1 to C10 alkyl group optionally containing an amide bond and/or an amino group, or R₁ and R₂ may bind to each other to form a C2 to 6 alkyl linker; and X₁, X₂, X₃, X₄, X₅, X₆, X₇ and X₈ identically or independently represent a bond or an aliphatic amino acid residue.

9. The PIPA according to claim 8, which is represented by the following Formula (Ia), (IIa), (IIIa) or (IVa):

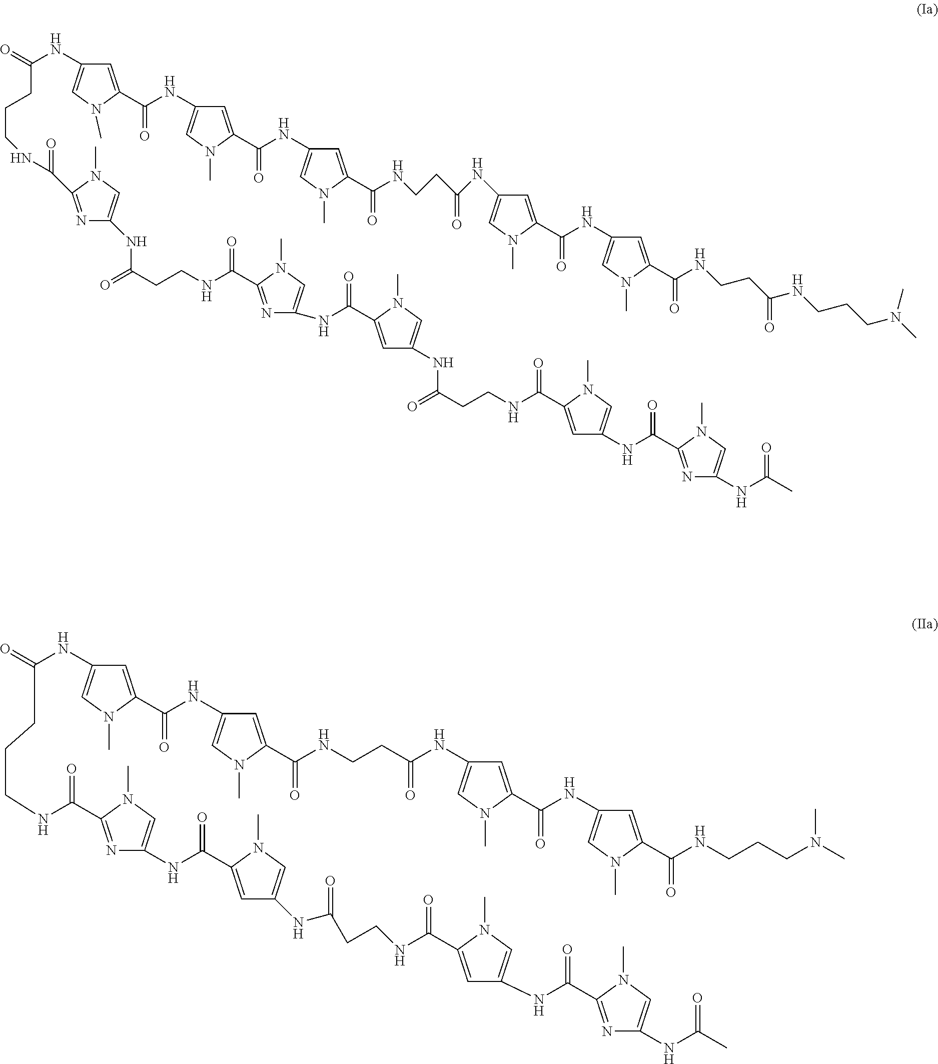 [SMILES: CC(=O)Nc1cn(C)c(C(=O)Nc2cc(C(=O)NCCC(=O)Nc3cc(C(=O)Nc4cn(C)c(C(=O)NCCC(=O)Nc5cn(C)c(C(=O)NCCCC(=O)Nc6cc(C(=O)Nc7cc(C(=O)Nc8cc(C(=O)NCCC(=O)Nc9cc(C(=O)Nc%10cc(C(=O)NCCC(=O)NCCCN(C)C)n(C)c%10)n(C)c9)n(C)c8)n(C)c7)n(C)c6)n5)n4)n(C)c3)n(C)c2)n1 ; CC(=O)Nc1cn(C)c(C(=O)Nc2cc(C(=O)NCCC(=O)Nc3cc(C(=O)Nc4cn(C)c(C(=O)NCCCC(=O)Nc5cc(C(=O)Nc6cc(C(=O)NCCC(=O)Nc7cc(C(=O)Nc8cc(C(=O)NCCCN(C)C)n(C)c8)n(C)c7)n(C)c6)n(C)c5)n4)n(C)c3)n(C)c2)n1]

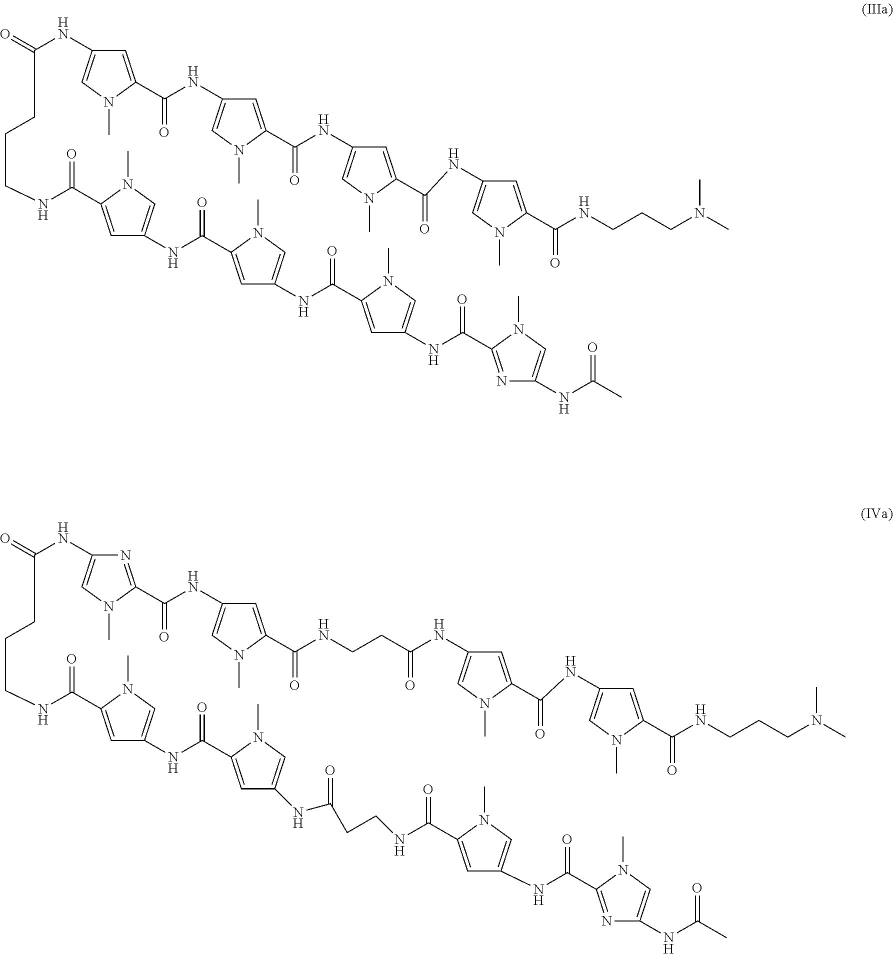 [SMILES: CC(=O)Nc1cn(C)c(C(=O)Nc2cc(C(=O)Nc3cc(C(=O)Nc4cc(C(=O)NCCCC(=O)Nc5cc(C(=O)Nc6cc(C(=O)Nc7cc(C(=O)Nc8cc(C(=O)NCCCN(C)C)n(C)c8)n(C)c7)n(C)c6)n(C)c5)n(C)c4)n(C)c3)n(C)c2)n1 ; CC(=O)Nc1cn(C)c(C(=O)Nc2cc(C(=O)NCCC(=O)Nc3cc(C(=O)Nc4cc(C(=O)NCCCC(=O)Nc5cn(C)c(C(=O)Nc6cc(C(=O)NCCC(=O)Nc7cc(C(=O)Nc8cc(C(=O)NCCCN(C)C)n(C)c8)n(C)c7)n(C)c6)n5)n(C)c4)n(C)c3)n(C)c2)n1]

10. A composition comprising the PIPA according to claim 1, wherein the PIPA inhibits the function of an IRF family transcription factor.

11. (canceled)

12. The composition according to claim 10, wherein the PIPA is represented by the following Formula (I), (II), (III) or (IV); 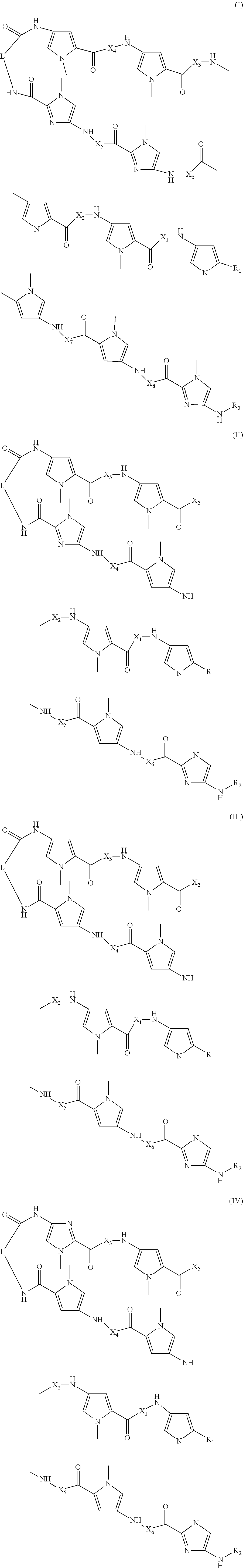
wherein in the Formulae (I), (II), (III) and (IV), L represents a C2 to 6 alkyl linker; R₁ and R₂ each represent a C1 to C10 alkyl group optionally having a substituent or a C1 to C10 alkyl group optionally containing an amide bond and/or an amino group, or R₁ and R₂ may bind to each other to form a C2 to 6 alkyl linker; and X₁, X₂, X₃, X₄, X₅, X₆, X₇ and X₈ identically or independently represent a bond or an aliphatic amino acid residue.

13-14. (canceled)

15. A method of inhibiting an IRF family transcription factor, comprising:
administering the PIPA according to claim 1 to a subject in need of inhibition of an IRF family transcription factor.

16. (canceled)

17. The method according to claim 15, wherein the PIPA is represented by the following Formula (I), (II), (III) or (IV): 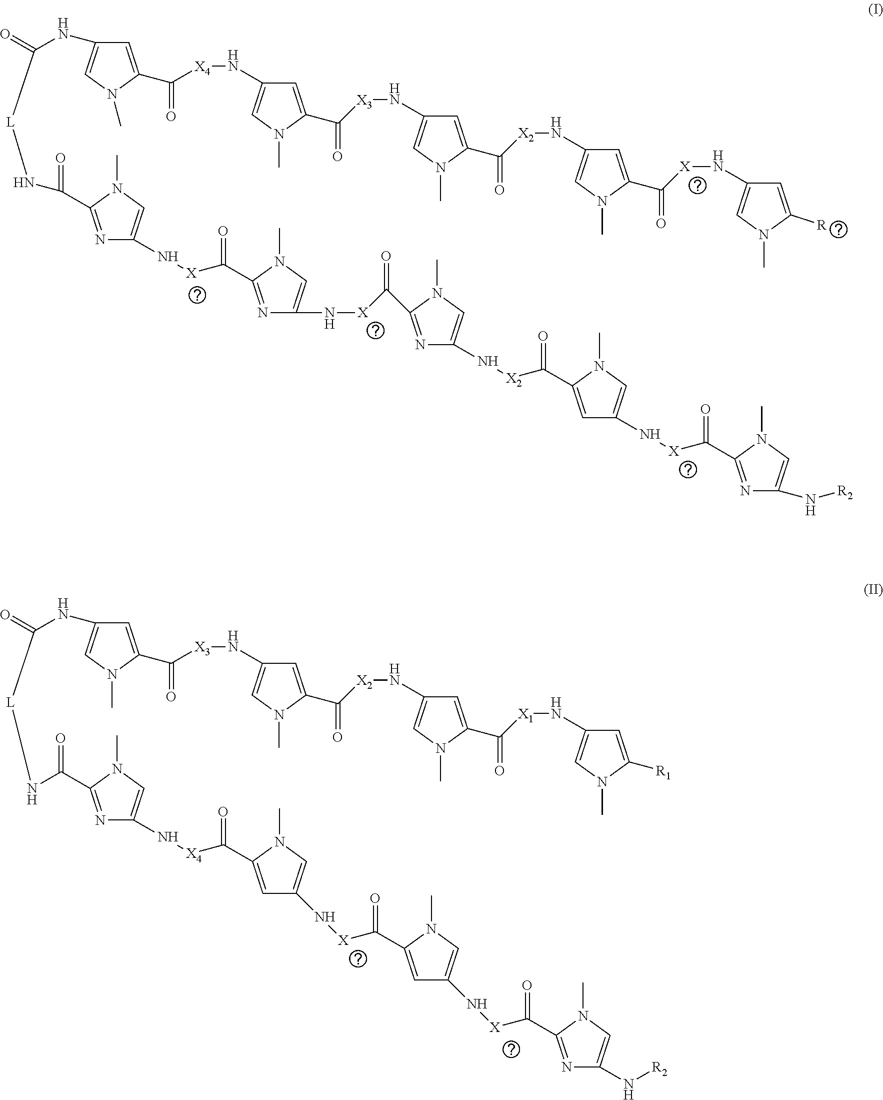 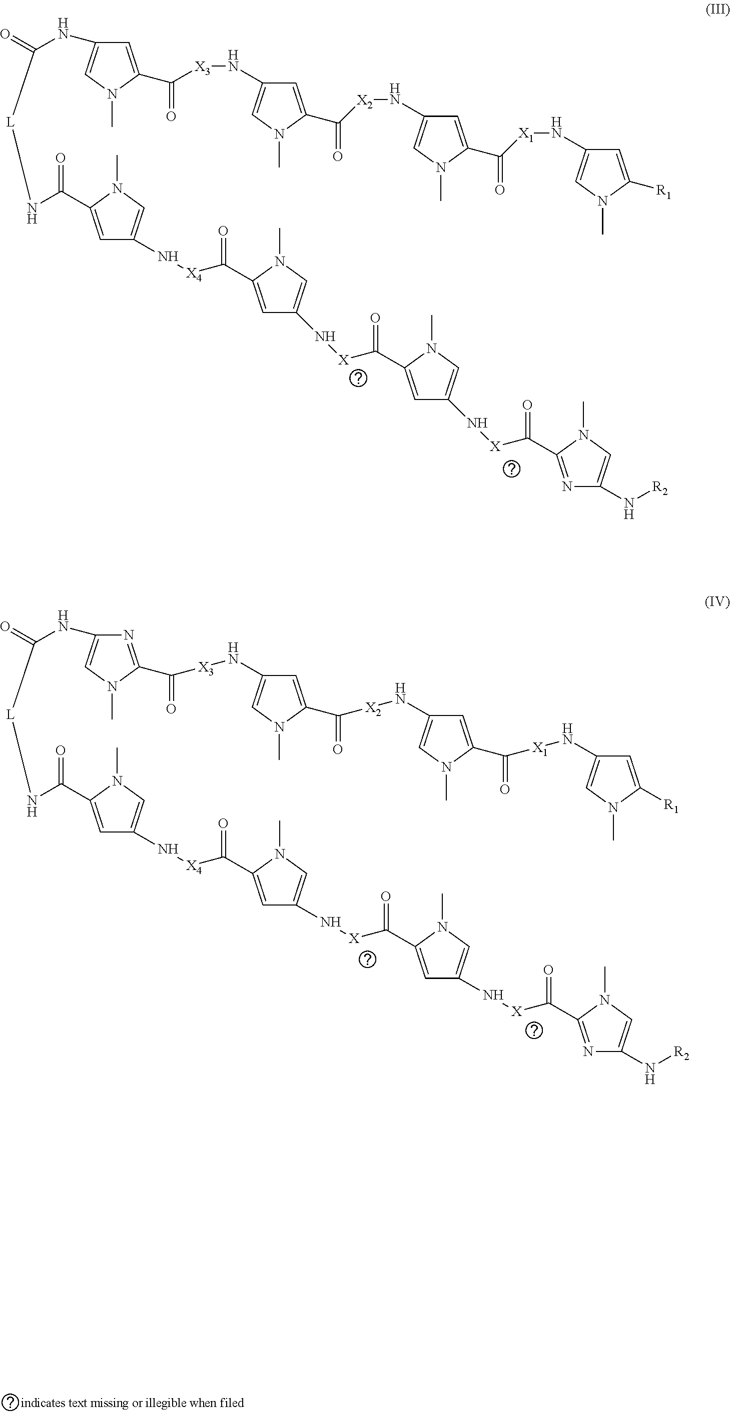
wherein in the Formulae (I), (II), (III) and (IV), L represents a C2 to 6 alkyl linker; R₁ and R₂ each represent a C1 to C10 alkyl group optionally having a substituent or a C1 to C10 alkyl group optionally containing an amide bond and/or an amino group, or R₁ and R₂ may bind to each other to form a C2 to 6 alkyl linker; and X₁, X₂, X₃, X₄, X₅, X₆, X₇ and X₈ identically or independently represent a bond or an aliphatic amino acid residue.

18. (canceled)

19. A medicament or a pharmaceutical composition for suppressing excessive immune response, comprising as an active ingredient, the PIPA according to claim 1.

20. (canceled)

21. The medicament or the pharmaceutical composition according to claim 19, wherein the PIPA is represented by the following Formula (I), (II), (III) or (IV): 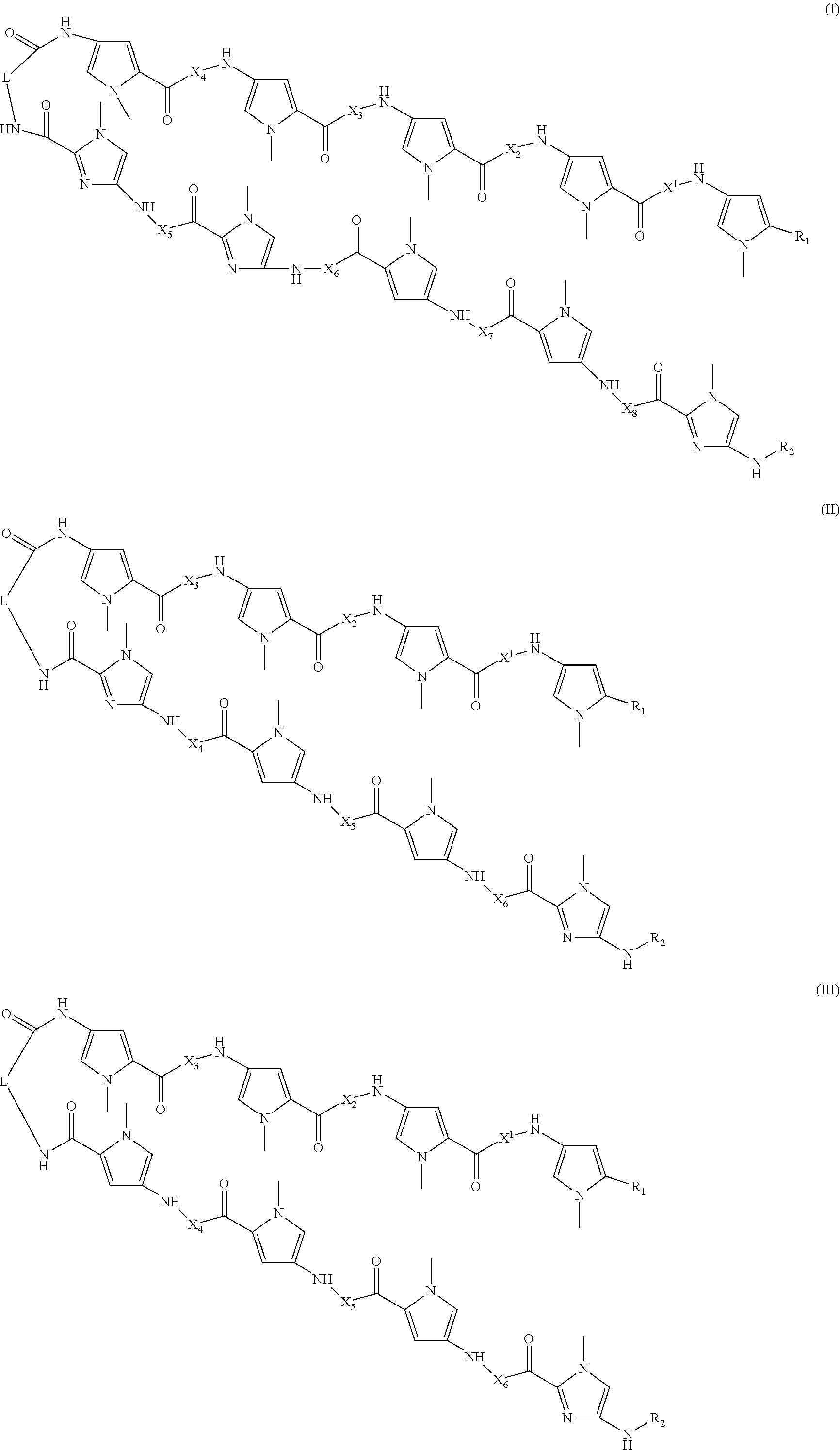 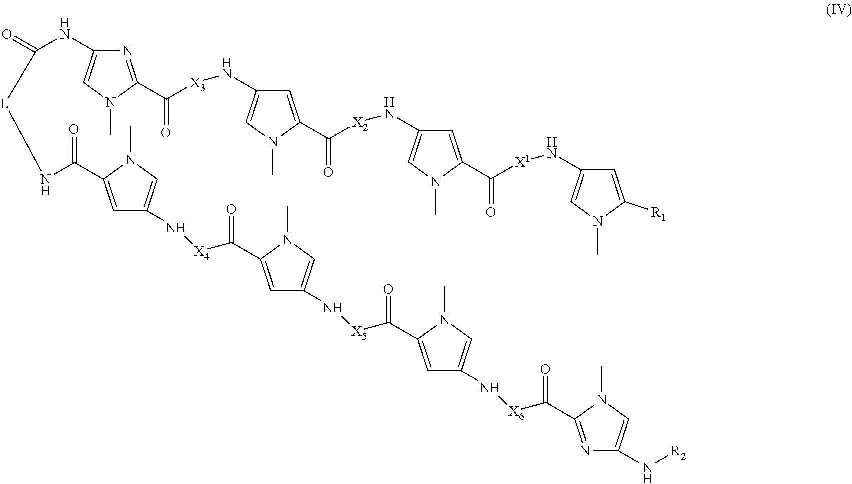
wherein in the Formulae (I), (II), (III) and (IV), L represents a C2 to 6 alkyl linker; R₁ and R₂ each represent a C1 to C10 alkyl group optionally having a substituent or a C1 to C10 alkyl group optionally containing an amide bond and/or an amino group, or R₁ and R₂ may bind to each other to form a C2 to 6 alkyl linker; and X₁, X₂, X₃, X₄, X₅, X₆, X₇ and X₈ identically or independently represent a bond or an aliphatic amino acid residue.

22. (canceled)

23. An adjuvant, comprising the PIPA according to claim 1.

24. (canceled)

25. The adjuvant according to claim 23, wherein the PIPA is represented by the following Formula (I), (II), (III) or (IV): 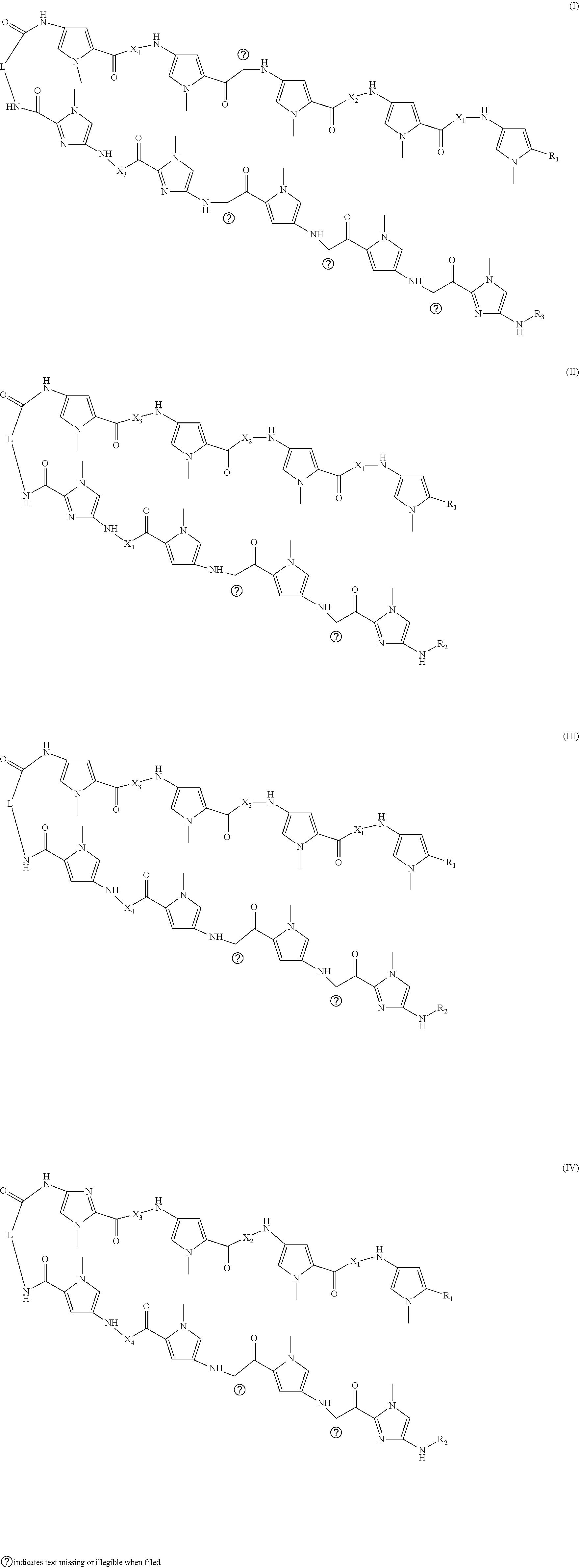
wherein in the Formulae (I), (II), (III) and (IV), L represents a C2 to 6 alkyl linker; R₁ and R₂ each represent a C1 to C10 alkyl group optionally having a substituent or a C1 to C10 alkyl group optionally containing an amide bond and/or an amino group, or R₁ and R₂ may bind to each other to form a C2 to 6 alkyl linker; and X₁, X₂, X₃, X₄, X₅, X₆, X₇ and X₈ identically or independently represent a bond or an aliphatic amino acid residue.

26. (canceled)

27. A method for producing an IRF family transcription factor inhibitor comprising a PIPA, comprising
providing the binding region of an IRF family transcription factor, and
producing a PIPA specifically binding to the binding region.

28. A conjugate, comprising the PIPA according to claim 1, and a PIPA auxiliary factor,
wherein said PIPA auxiliary factor is a substance different from said PIPA, and
wherein said PIPA auxiliary factor enhances the function of said PIPA, and/or assists the action of the PIPA.

29-32. (canceled)

33. The conjugate according to claim 28, wherein the PIPA is represented by the following Formula (I), (II), (III) or (IV): 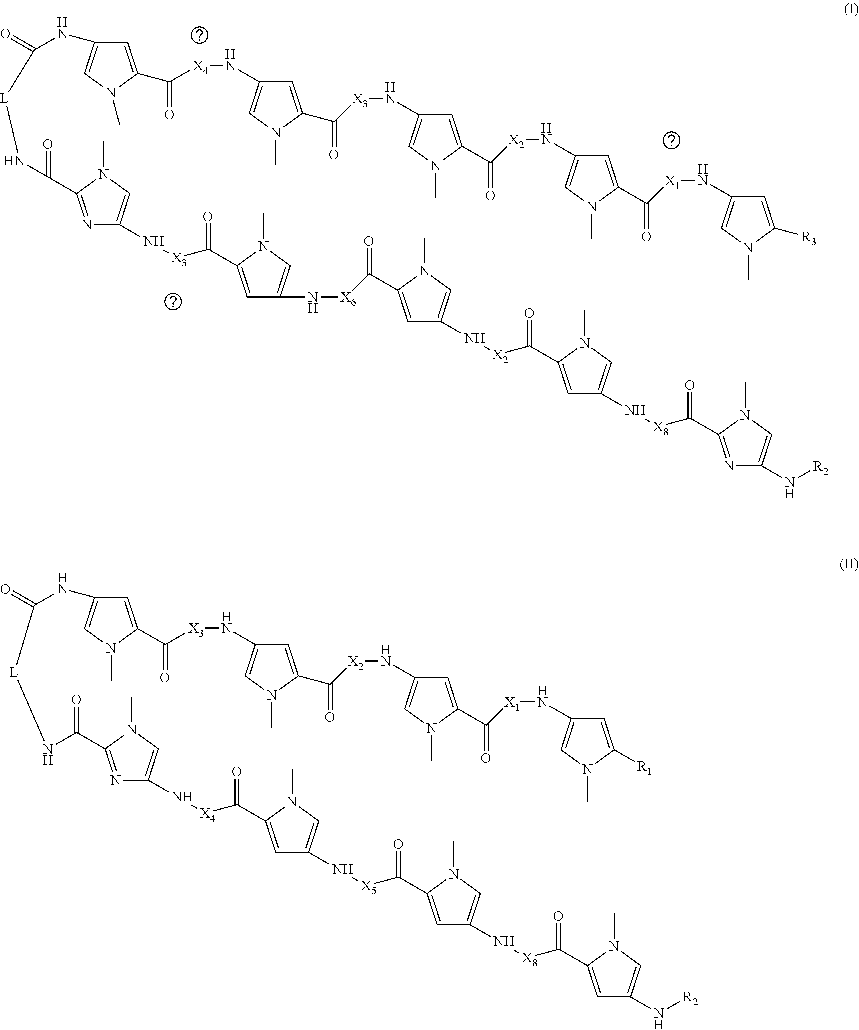 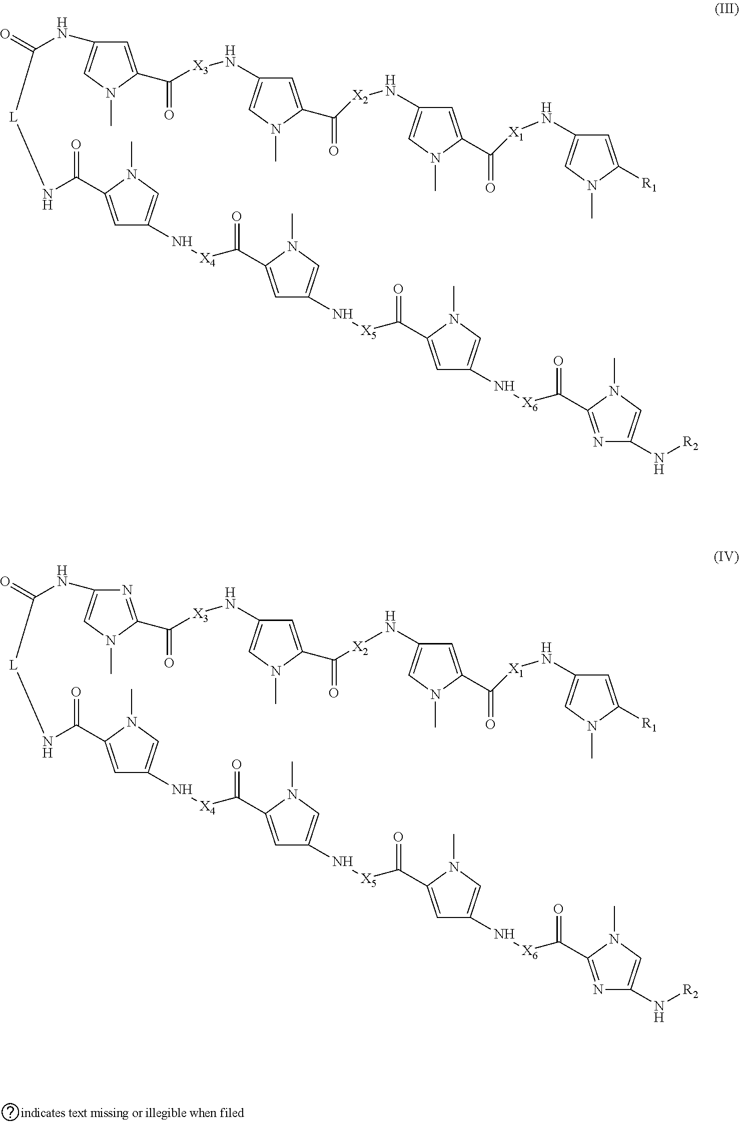
wherein in the Formulae (I), (II), (III) and (IV), L represents a C2 to 6 alkyl linker; R₁ and R₂ each represent a C1 to C10 alkyl group optionally having a substituent or a C1 to C10 alkyl group optionally containing an amide bond and/or an amino group, or R₁ and R₂ may bind to each other to form a C2 to 6 alkyl linker; and X₁, X₂, X₃, X₄, X₅, X₆, X₇ and X₈ identically or independently represent a bond or an aliphatic amino acid residue.

34-38. (canceled)


SEQUENCE LISTING (28 sequences)

SEQ ID NO: 1-27 - skipped (27 entries)

SEQ ID NO: 28 - DNA, 24 bp, synthetic construct
  misc_feature 1..24: note = Sequence used for the binding analysis of PIPA
  source 1..24: mol_type = other DNA
gaaagtgcgaaagtgcgaaagtgc